(19) United States
(12) Patent Application Publication
(10) Pub. No.: US 2008/0292547 A1
(43) Pub. Date: Nov. 27, 2008

(54) Novel Imaging Agents for Fibrosis

(76) Inventors: Helge Tolleshaug, Oslo (NO); Ben Newton, Buckinghamshire (GB); Anna Rydbeck, Staffanstorp (SE); Salah Chettibi, Buckinghamshire (GB); Morten Eriksen, Oslo (NO)

Correspondence Address: GE HEALTHCARE, INC., IP DEPARTMENT, 101 CARNEGIE CENTER, PRINCETON, NJ, 08540-6231, US

(21) Appl. No.: 12/096,387

(22) PCT Filed: Dec. 7, 2006
(86) PCT No.: PCT/GB2006/004579

§ 371 Date: Jun. 6, 2008

(30) Foreign Application Priority Data
Dec. 8, 2005 (GB) 0524991.7

(51) Int. Cl. A61K 49/00; A61K 51/04

(52) U.S. Cl. 424/1.65; 424/9.1

(57) ABSTRACT

The present invention provides a novel imaging agent suitable for the non-invasive visualization of fibrosis. A method for the preparation of the imaging agent is also provided by the invention, as well as a precursor for use in said method. Also provided is a pharmaceutical composition comprising the imaging agent and a kit for the preparation of the pharmaceutical. In a further aspect, use of the imaging agent for in vivo imaging and in the preparation of a medicament for the diagnosis of a condition in which LOX is upregulated is provided.

### TECHNICAL FIELD OF THE INVENTION

The present invention relates to diagnostic imaging and in particular to the diagnostic imaging of fibrosis. Diagnostic imaging agents are described which are suitable for this purpose, particularly for the diagnostic imaging of fibrosis in the liver, heart, kidneys and lungs.

### DESCRIPTION OF RELATED ART

Fibrosis is a process characterized by the excessive secretion of extracellular matrix components. This is caused by increased synthesis and decreased degradation of matrix proteins, most notably collagen types I and III, and is triggered as a response to tissue damage resulting from inflammation, infection or injury. In simple terms, fibrosis is scar tissue and forms part of all “repair” processes in tissue. However, because of ongoing inflammation, infection and repeated injury, fibrosis scar tissue builds up and does not replace “functional” cells, thus leading to abnormal organ function and eventually organ failure.

Fibrosis is one of the major, classic pathological processes in medicine. It is a key component of multiple diseases that affect millions of people worldwide including:
a) Lung diseases such as idiopathic pulmonary fibrosis (lung fibrosis of unknown origin), asthma and chronic obstructive pulmonary disease
b) Scleroderma: a heterogeneous and life threatening disease characterised by the excessive extracellular matrix deposition within connective tissue of the body (i.e. Skin and visceral organs).
c) Post-surgical scarring following transplantation
d) Diabetic retinopathy and age-related macular degeneration (fibrotic diseases of the eye and leading causes of blindness)
e) Cardiovascular disease including atherosclerosis and vulnerable plaque
f) Kidney fibrosis linked to diabetes—diabetic nephropathy and glomerulosclerosis
g) IgA nephropathy (causes of kidney failure and the need for dialysis and retransplant)
h) Cirrhosis and biliary atresia (leading causes of liver fibrosis and failure)
i) Rheumatoid arthritis
j) Autoimmune diseases such as dermatomyositis
k) Congestive heart failure

The clinical manifestations of fibrosis vary widely. Taking the example of cirrhosis, the clinical manifestations vary from no symptoms at all, to liver failure, and are determined by both the nature and severity of the underlying liver disease as well as the extent of hepatic fibrosis. Up to 40% of patients with cirrhosis are asymptomatic and may remain so for more than a decade, but progressive deterioration is inevitable once complications develop including ascites, variceal hemorrhage or encephalopathy. Fibrosis and cirrhosis therefore represent the consequences of a sustained wound healing response to chronic liver injury from a variety of causes including viral, autoimmune, drug induced, cholestatic and metabolic diseases. The common causes of liver fibrosis and cirrhosis include immune mediated damage, genetic abnormalities, and non-alcoholic steatohepatitis (NASH), which is particularly associated with diabetes and metabolic syndrome (MS). There is a high incidence of MS in the western population. This syndrome typically occurs in individuals who are obese, have hyperlipidemia and hypertension, and often leads to the development of type II diabetes. The hepatic manifestation of metabolic syndrome is non-alcoholic fatty liver disease (NAFLD), with an estimated prevalence in the USA of 24% of the population. A fatty liver represents the less severe end of a spectrum of NAFLD that may progress to NASH and ultimately to cirrhosis of the liver. The development of fibrosis demonstrates a risk of such progression, and is presently assessed by means of a liver biopsy. However, liver biopsy causes significant discomfort, is not without risk and is costly. Furthermore, available blood tests for hepatic fibrosis are not reliable in NAFLD.

The strength of collagen is provided by crosslinking between various lysine residues both within a fibril and between fibrils. The first step of the crosslinking process is the deamination of lysine and hydroxylysine residues by lysyl oxidase enzymes (LOX) to produce aldehyde groups. These highly reactive groups then form the crosslinks. A number of patent documents discuss the use of LOX binders for the treatment of fibrotic disease.

WO 96/040746 describes anti-fibrotic agents useful in controlling or treating various pathologic fibrotic disorders or abnormalities. Homocysteine thiolactone and analogues thereof were demonstrated to inhibit LOX activity with IC₅₀ values of between 4 and 25 μM.

WO 03/097612 describes 2-phenyl-3(2H)-pyridazinones useful in the treatment of fibrotic diseases. The compounds described in the patent application are demonstrated to inhibit LOX activity with IC₅₀ values of 0.005-0.07 μM.

U.S. Pat. No. 5,252,608 describes a method of treating diseases associated with the abnormal deposition of collagen using halogenated allylamines. These compounds were demonstrated to inhibit LOX activity with IC₅₀ values of between 0.0001 and 1 μM.

U.S. Pat. No. 4,997,854 describes a class of diamine anti-fibrotic agents that act as analogue substrate inhibitors of lysyl oxidase and have use in the treatment of fibrotic disease. Micromolor IC50 values were reported for some specific compounds.

None of the above mentioned prior art documents suggest use of LOX binders as diagnostic imaging agents. A need therefore exists for a non-invasive test for the detection of fibrosis and in particular liver fibrosis.

### SUMMARY OF THE INVENTION

The present invention provides a novel imaging agent suitable for the non-invasive visualization of fibrosis. A method for the preparation of the imaging agent is also provided by the invention, as well as a precursor for use in said method. Also provided is a pharmaceutical composition comprising the imaging agent and a kit for the preparation of the pharmaceutical. In a further aspect, use of the imaging agent for in vivo imaging and in the preparation of a medicament for the diagnosis of a condition in which LOX is upregulated is provided.

### DETAILED DESCRIPTION OF THE INVENTION

In one aspect, the present invention provides an imaging agent comprising:
(i) a lysyl oxidase (LOX) binder; and,
(ii) an imaging moiety
wherein said imaging moiety is either an integral part of the LOX binder or is conjugated to the LOX binder via a suitable chemical group.

By the term “imaging agent” is meant a compound designed to target a particular physiology or pathophysiology in a mammal, and which can be detected following its administration to the mammalian body in vivo.

In the imaging agent of the invention, the imaging moiety may be present as an integral part of the LOX binder, e.g. one of the atoms of the LOX binder could be ¹¹C instead of ¹²C. Alternatively, the imaging moiety may be conjugated to the LOX binder via a suitable chemical group, e.g. a metal chelate which can complex an imaging moiety which is a metal ion. A linker may also be present linking the LOX binder to either the suitable chemical group or directly to the imaging moiety itself. Suitable linkers of the present invention are of Formula -(L¹)ₙ- wherein:
each L¹ is independently —CO—, —CR′₂—, —CR′═CR′—, —C≡C—, —CR′₂CO₂—, —CO₂CR′₂—, —NR′—, —NR′CO—, —CONR′—, —NR′(C═O)NR′—, —NR′(C═S)NR′—, —SO₂NR′—, —NR′SO₂—, —CR′₂OCR′₂—, —CR′₂SCR′₂—, —CR′₂NR′CR′₂—, a C₄₋₈ cycloheteroalkylene group, a C₄₋₈ cycloalkylene group, a C₅₋₁₂ arylene group, a C₃₋₁₂ heteroarylene group, an amino acid, a polyalkyleneglycol, polylactic acid or polyglycolic acid moiety;
n is an integer of value 0 to 15;
each R′ group is independently H or C₁₋₁₀ alkyl, C₃₋₁₀ alkylaryl, C₂₋₁₀ alkoxyalkyl, C₁₋₁₀ hydroxyalkyl, C₁₋₁₀ fluoroalkyl, or 2 or more R′ groups, together with the atoms to which they are attached form a carbocyclic, heterocyclic, saturated or unsaturated ring.

It is envisaged that branched linker groups are also possible, i.e. a linker group -(L¹)ₙ-substituted with a further linker group -(L²)ₒ-, which terminates with an R″ group wherein L², o and R″ are as defined respectively for L¹, n and R′ above.

Such linkers are particularly useful in the context of manipulating the biodistribution and/or excretion profiles of the imaging agent. For example, the inclusion of a linker comprising polyethylene glycol groups or acetyl groups can improve the blood residence time of the imaging agent.

By the term “amino acid” is meant an L- or D-amino acid, amino acid analogue (e.g. napthylalanine) or amino acid mimetic which may be naturally occurring or of purely synthetic origin, and may be optically pure, i.e. a single enantiomer and hence chiral, or a mixture of enantiomers. Preferably the amino acids of the present invention are optically pure.

Such linkers also have application in relation to other parts of the invention as described below. For this application, preferred L¹ and L² groups are —CO—, —CH₂—, —NH—, —NHCO—, —CONH—, —CH₂OCH₂—, and amino acid residues.

The term “lysyl oxidase (LOX) binder” in the context of the present invention is taken to mean a compound capable of binding to LOX in vitro with a Kd value of less than 100 nM, preferably less than 50 nM and most preferably less than 10 nM. In a preferred embodiment, the LOX binder is capable of inhibiting the enzyme activity of LOX in vitro [e.g. as described in WO 96/040746] at IC₅₀ values of less than 10 μM, preferably less than 1 μM, most preferably less than 0.1 μM and especially preferably less than 0.01 μM.

Preferably, the LOX binder is selected from:
(i) a homocysteine lactone;
(ii) a pyridazinone;
(iii) a halogenated allylamine;
(iv) a vicinal diamine; and
(v) β-aminoproprionitrile and derivatives thereof.

Most preferably, the LOX binder is a homocysteine lactone, a halogenated allylamine or a vicinal diamine.

Where said LOX binder is a homocysteine lactone, it is preferably of Formula I:

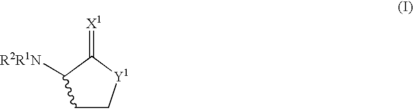

wherein
R¹ and R² are independently selected from the group consisting of hydrogen, an amino acid, C₁₋₆ alkyl, halo, C₁₋₆ haloalkyl, hydroxyl, C₁₋₆ hydroxyalkyl, C₁₋₆ alkoxyl, C₂₋₆ alkoxyalkyl, C₁₋₆ acyl, C₂₋₆ alkacyl, C₁₋₆ carboxyl, C₂₋₆ carboxyalkyl, amino, C₁₋₆ alkylamino, nitro, cyano, and thiol;
X¹ and Y¹ are independently selected from S, Se or O.

Preferably for Formula I:

R¹ and R² are independently selected from the group consisting of hydrogen, an amino acid, C₁₋₆ alkyl, C₁₋₆ haloalkyl, C₁₋₆ hydroxyalkyl, C₂₋₆ alkoxyalkyl, C₂₋₆ carboxyalkyl or C₁₋₆ alkylamino.

Most preferably for Formula I:

R¹ is hydrogen and R² is an amino acid, or C₁₋₆ alkylamino.

Some examples of preferred homocysteine lactones of the present invention are:
(i) glycylhomocysteine thiolactone
(ii) β-alanylhomocysteine thiolactone
(iii) γ-aminobutyrylhomocysteine thiolactone
(iv) ε-aminocaproylhomocysteine thiolactone
(v) lysylhomocysteine thiolactone

A method for the synthesis of the above preferred homocysteine lactones is described in WO 96/040746.

Where said LOX binder is a pyridazinone, it is preferably of Formula II:

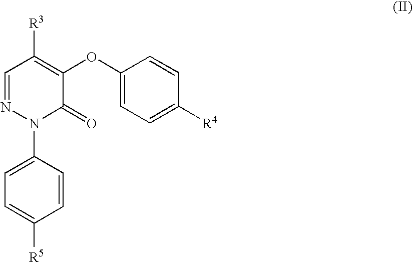

wherein:
one of R³ and R⁴ is X² and the other is Y² wherein;
X² is a substituted 5- or 6-membered nitrogen-containing aliphatic or aromatic ring substituted with 0-4 substituents selected from C₁₋₆ alkyl, C₁₋₆ hydroxyalkyl, C₁₋₆ sulphonyl and imidazolyl; and,
Y² is a phenyl group substituted with 0-4 substituents selected from C₁₋₆ alkyl, hydroxyl, halo, C₁₋₆ aminoalkyl, and C₁₋₆ alkylamido;
   R⁵ is methyl or chloro.

Preferably for Formula II:

X² is pyrroyl, imidazoyl, pyrazoyl, piperidyl or piperazinyl substituted with 0-2 substituents selected from C₁₋₆ alkyl, C₁₋₆ hydroxyalkyl and C₁₋₆ sulphonyl.

Most preferably for Formula II:

X² is imidazoyl, piperidyl or piperazinyl substituted with 0-2 substituents selected from C₁₋₆ alkyl, C₁₋₆ hydroxyalkyl and C₁₋₆ sulphonyl; and,
Y² a phenyl group substituted with 0-2 substituents selected from hydroxyl, fluoro, C₁₋₆ aminoalkyl and carbamoyl.

Examples of preferred pyridazinones of the invention are:

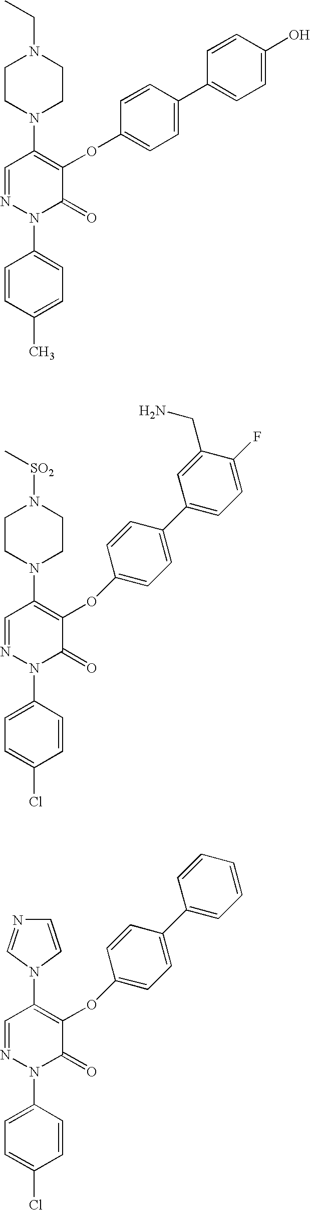

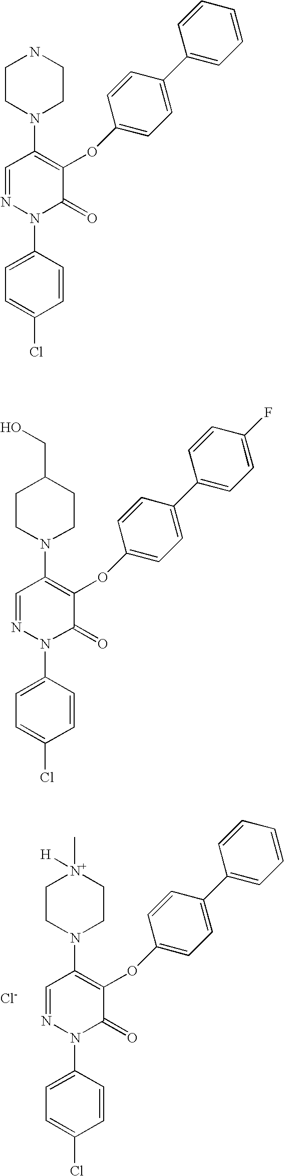

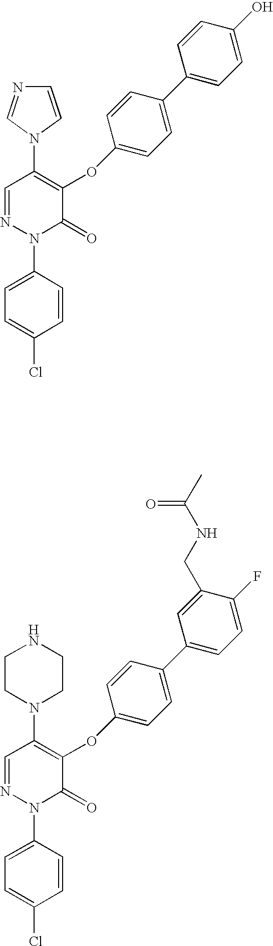

A method for the synthesis of the above pyridazinones is described in WO 03/097612.

Where said LOX binder is a halogenated allylamine, it is preferably of Formula III:

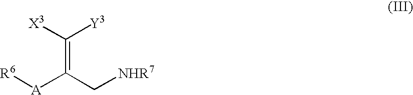

wherein:
R⁶ is methyl, naphthyl, indenyl, fluorenyl, piperidinyl, pyrrolyl, thienyl, furanyl, indolyl, thianophthylenyl, benzofuronyl, or a phenyl group substituted with 0-4 substituents selected from C₁₋₆ alkyl, C₁₋₆ alkoxy, hydroxyl, chloro, fluoro, bromo, iodo, trifluoromethyl, nitro, C₂₋₆ alkylcarbonyl, benzoyl or phenyl;
R⁷ is hydrogen or C₁₋₆ alkyl;
A is a linker of Formula -(L³)ₚ- wherein L³ and p are as previously described for L¹ and n; and,
X³ and Y³ are independently selected from the group consisting of hydrogen, fluoro, chloro and bromo

Preferably for Formula III:

R⁶ is a phenyl group substituted with 0-2 substituents selected from C₁₋₆ alkyl, C₁₋₆ alkoxy, hydroxyl, chloro, fluoro, bromo, iodo, trifluoromethyl, nitro, C₂₋₆ alkylcarbonyl, benzoyl or phenyl;
R⁷ is hydrogen;
A is —(CH₂)_{q}— wherein q is in the range 1-6; and,
X³ is hydrogen.

Most preferably for Formula III:

R⁶ is a phenyl group substituted with 0-2 substituents selected chloro, fluoro, bromo and iodo;
R⁷ is hydrogen;
A is —(CH₂)_{q}— wherein q is in the range 1-6; and,
X³ is hydrogen and Y³ is fluoro.

Due to the presence of one or two double bonds in the compounds of Formula III geometric isomerism is possible, i.e. at the allyl amine double bond and also potentially in the A group. Substantially pure isomers and mixtures of isomers are covered by the present invention. In compounds where one of X³ and Y³ is a halogen and the other a hydrogen, the halogen is preferably orientated cis to the -A-R⁶ group.

An examples of a halogenated allylamine of the invention is:

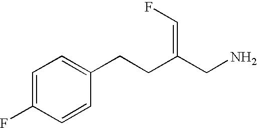

A method for the synthesis of the above halogenated allylamine is described in U.S. Pat. No. 5,252,608.

Where said LOX binder is a vicinal diamine, it is preferably of Formula IV:

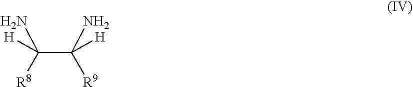

wherein R⁸ and R⁹ are each independently hydrogen, C₁₋₆ alkyl, or R⁸ and R⁹ together with the carbons to which they are attached form a 6-14-membered optionally-substituted aliphatic or aromatic ring system.

It is preferred that the two primary amine groups of Formula IV are aligned in the some stereochemical plane. Therefore, when the vicinal diamine is an unsaturated or a cyclic structure, the molecular configuration should assume a cis orientation rather than a trans orientation.

A method for the synthesis of vicinal diamines of Formula IV is outlined in Gacheru et al [1989 J. Biol. Chem. 264(22) pp. 12963-9], as well as in U.S. Pat. No. 4,997,854.

Preferably, for compounds of Formula IV, R⁸ and R⁹ together with the carbons to which they are attached form substituted cyclohexyl or substituted dicyclohexyl wherein the substituents are preferably selected from C₁₋₃ alkyl and halo.

Most preferred compounds of Formula IV are compounds of Formulae IVa and IVb as follows:

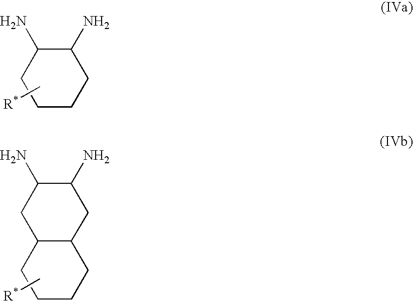

wherein R* is methyl, methoxy, chloro, fluoro or bromo.

The “imaging moiety” may be detected either external to the human body or via use of detectors designed for use in vivo, such as intravascular radiation or optical detectors such as endoscopes, or radiation detectors designed for intra-operative use.

The imaging moiety is preferably chosen from:
(i) a radioactive metal ion;
(ii) a paramagnetic metal ion;
(iii) a gamma-emitting radioactive halogen;
(iv) a positron-emitting radioactive non-metal;
(v) a hyperpolarised NMR-active nucleus;
(vi) a reporter suitable for in vivo optical imaging;
(vii) a β-emitter suitable for intravascular detection.

When the imaging moiety is a radioactive metal ion, i.e. a radiometal, suitable radiometals can be either positron emitters such as ⁶⁴Cu, ⁴⁸V, ⁵²Fe, ⁵⁵Co, ^{94m}Tc or ⁶⁸Ga; γ-emitters such as ^{99m}Tc, ¹¹¹In, ^{113m}In, or ⁶⁷Ga. Preferred radiometals are ^{99m}Tc, ⁶⁴Cu, ⁶⁸Ga and ¹¹¹In. Most preferred radiometals are γ-emitters, especially ^{99m}Tc.

When the imaging moiety is a paramagnetic metal ion, suitable such metal ions include: Gd(III), Mn(II), Cu(II), Cr(III), Fe(III), Co(II), Er(II), Ni(II), Eu(III) or Dy(III). Preferred paramagnetic metal ions are Gd(III), Mn(II) and Fe(III), with Gd(III) being especially preferred.

When the imaging moiety is a gamma-emitting radioactive halogen, the radiohalogen is suitably chosen from ¹²³I, ¹³¹I or ⁷⁷Br. ¹²⁵I is specifically excluded as it is not suitable for use as an imaging moiety for diagnostic imaging. A preferred gamma-emitting radioactive halogen is ¹²³I.

When the imaging moiety is a positron-emitting radioactive non-metal, suitable such positron emitters include: ¹¹C, ¹³N, ¹⁵O, ¹⁷F, ¹⁸F, ⁷⁵Br, ⁷⁶Br or ¹²⁴I. Preferred positron-emitting radioactive non-metals are ¹¹C, ¹³N, ¹⁸F and ¹²⁴I, especially ¹¹C and ¹⁸F, most especially ¹⁸F.

When the imaging moiety is a hyperpolarised NMR-active nucleus, such NMR-active nuclei have a non-zero nuclear spin, and include ¹³C, ¹⁵N, ¹⁹F, ²⁹Si and ³¹P. Of these, ¹³C is preferred. By the term “hyperpolarised” is meant enhancement of the degree of polarisation of the NMR-active nucleus over its' equilibrium polarisation. The natural abundance of ¹³C (relative to ¹²C) is about 1%, and suitable ¹³C-labelled compounds are suitably enriched to an abundance of at least 5%, preferably at least 50%, most preferably at least 90% before being hyperpolarised. At least one carbon atom of the imaging agent of the invention is suitably enriched with ¹³C, which is subsequently hyperpolarised.

When the imaging moiety is a reporter suitable for in vivo optical imaging, the reporter is any moiety capable of detection either directly or indirectly in an optical imaging procedure. The reporter might be a light scatterer (e.g. a coloured or uncoloured particle), a light absorber or a light emitter. More preferably the reporter is a dye such as a chromophore or a fluorescent compound. The dye can be any dye that interacts with light in the electromagnetic spectrum with wavelengths from the ultraviolet light to the near infrared. Most preferably the reporter has fluorescent properties.

Preferred organic chromophoric and fluorophoric reporters include groups having an extensive delocalized electron system, e.g. cyanines, merocyaonines, indocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthroquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes. Fluorescent proteins, such as green fluorescent protein (GFP) and modifications of GFP that have different absorption/emission properties are also useful. Complexes of certain rare earth metals (e.g., europium, samarium, terbium or dysprosium) are used in certain contexts, as are fluorescent nanocrystals (quantum dots).

Particular examples of chromophores which may be used include: fluorescein, sulforhodamine 101 (Texas Red), rhodamine B, rhodamine 6G, rhodamine 19, indocyanine green, Cy2, Cy3, Cy3.5, Cy5, Cy5.5, Cy7, Marina Blue, Pacific Blue, Oregon Green 88, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750.

Particularly preferred are dyes which have absorption maxima in the visible or near infrared (NIR) region, between 400 nm and 3 μm, particularly between 600 and 1300 nm. Optical imaging modalities and measurement techniques include, but not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarisation, luminescence, fluorescence lifetime, quantum yield, and quenching.

When the imaging moiety is a β-emitter suitable for intravascular detection, suitable such β-emitters include the radiometals ⁶⁷Cu, ⁸⁹Sr, ⁹⁰Y, ¹⁵³Sm, ¹⁸⁶Re, ¹⁸⁸Re or ¹⁹²Ir, and the non-metals ³²P, ³³P, ³⁸S, ³⁸Cl, ³⁹Cl, ⁸²Br and ⁸³Br.

Preferred imaging moieties are those which can be detected externally in a non-invasive manner following administration in viva. Most preferred imaging moieties are radioactive, especially radioactive metal ions, gamma-emitting radioactive halogens and positron-emitting radioactive non-metals, particularly those suitable for imaging using SPECT or PET.

Preferred imaging agents of the invention do not undergo facile metabolism in viva, and hence most preferably exhibit a half-life in vivo of 60 to 240 minutes in humans. The imaging agent is preferably excreted via the kidney (i.e. exhibits urinary excretion). The imaging agent preferably exhibits a signal-to-background ratio at diseased foci of at least 1.5, most preferably at least 5, with at least 10 being especially preferred. Where the imaging agent comprises a radioisotope, clearance of one half of the peak level of imaging agent which is either non-specifically bound or free in vivo, preferably occurs over a time period less than or equal to the radioactive decay half-life of the radioisotope of the imaging moiety.

Furthermore, the molecular weight of the imaging agent is suitably up to 5000 Daltons. Preferably, the molecular weight is in the range 150 to 3000 Daltons, most preferably 200 to 1500 Daltons, with 300 to 800 Daltons being especially preferred.

In a preferred embodiment, where the LOX binder is a homocysteine lactone of Formula I, the imaging moiety is an integral part of either R¹ or R², most preferably R², for example:

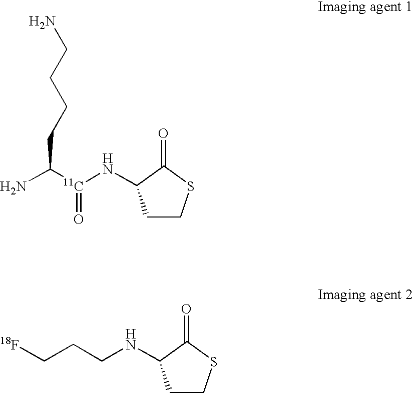

In an alternative preferred embodiment where the LOX binder is a homocysteine lactone of Formula I, the imaging moiety is conjugated to R¹ or R², most preferably to R², either directly or via a suitable chemical group and/or linker, for example:

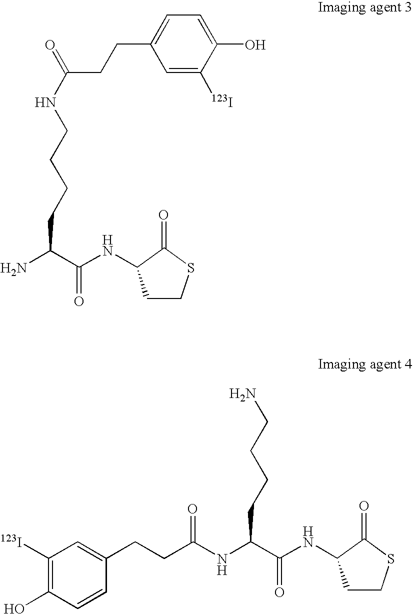

Synthetic routes for obtaining imaging agents 3 and 4 are described in Examples 4 and 5.

In a preferred embodiment where the LOX binder is a pyridazinone of Formula II, the imaging moiety may be an integral part of either R³ or R⁴, most preferably R⁴, for example:

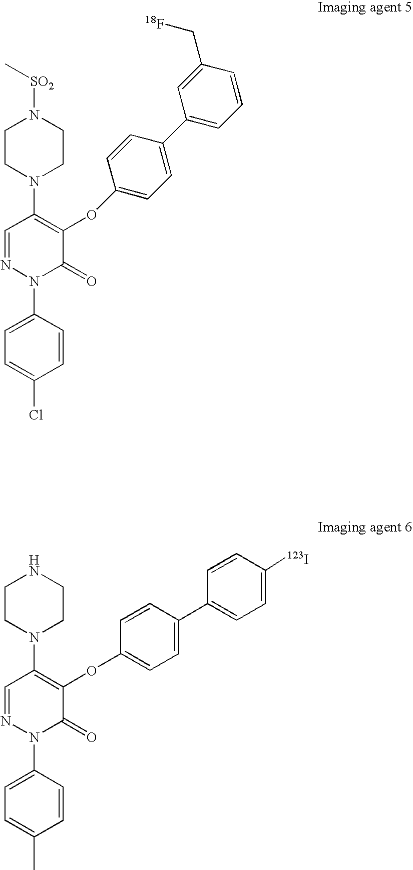

In an alternative preferred embodiment where the LOX binder is a pyridazinone of Formula II, the imaging moiety may be conjugated to R³ or R⁴, preferably to R⁴, either directly or via a suitable chemical group and/or linker, for example:

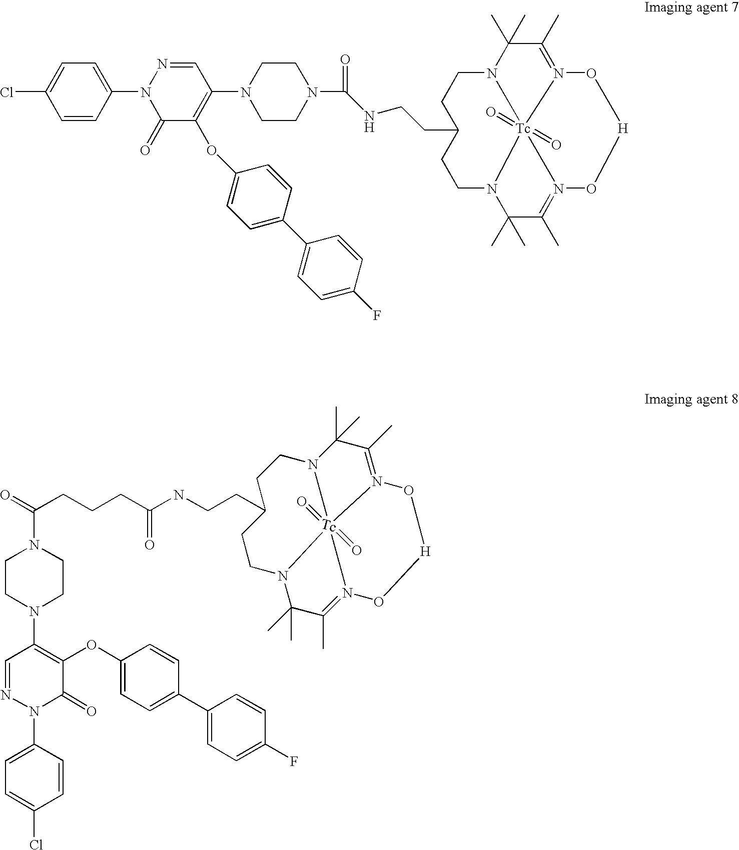

where Tc is ^{99m}Tc.

In a preferred embodiment where the LOX binder is a halogenated allylamine of Formula III, the imaging moiety may be an integral part of R⁶, R⁷ or Y², most preferably R⁷, for example:

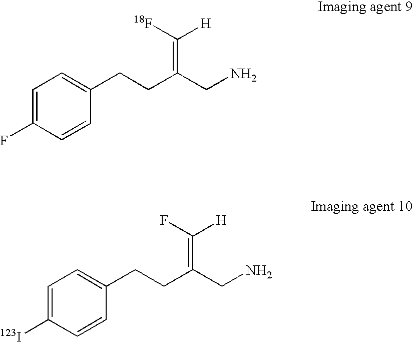

Synthesis of a non-radioactive version of Imaging agent 9 is described in Example 11.

In an alternative preferred embodiment where the LOX binder is a halogenated allylamine of Formula III, the imaging moiety may be conjugated to the R⁶, R⁷ or Y², most preferably R⁷, either directly or via a suitable chemical group and/or linker, for example:

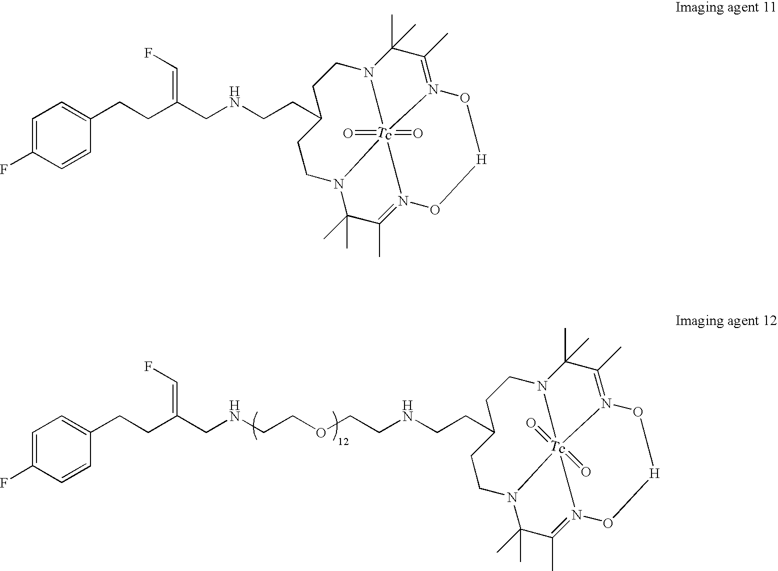

In a preferred embodiment, where the LOX binder is a vicinal diamine of Formula IV, the imaging moiety is conjugated at R⁸ and/or R⁹, for example:

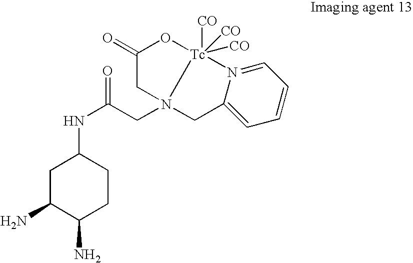

Synthesis of the above compound is described in Example 10.

Synthesis of the imaging agents via precursor compounds is described in more detail below in relation to a further aspect of the invention.

In a further aspect, the present invention provides a method for the preparation of the imaging agent of the invention comprising reaction of a precursor with a suitable source of an imaging moiety wherein said precursor comprises:
(i) a LOX binder as defined previously; and,
(ii) a chemical group capable of reacting with a source of the imaging moiety to give the imaging agent of the invention;
   wherein said chemical group is either an integral part of said LOX binder or is conjugated to said LOX binder.

A “precursor” comprises a derivative of the LOX binder of the invention, designed so that chemical reaction with a convenient chemical form of the imaging moiety occurs site-specifically; can be conducted in the minimum number of steps (ideally a single step); and without the need for significant purification (ideally no further purification), to give the desired imaging agent. Such precursors are synthetic and can conveniently be obtained in good chemical purity. The “precursor” may optionally comprise a protecting group for certain functional groups of the LOX binder.

By the term “protecting group” is meant a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Protecting groups are well known to those skilled in the art and are suitably chosen from, for amine groups: Boc (where Boc is tert-butyloxycarbonyl), Fmoc (where Fmoc is fluorenylmethoxycarbonyl), trifluoroacetyl, allyloxycarbonyl, Dde [i.e. 1-(4,4-dimethyl-2,6-dioxocyclohexylidene)ethyl] or Npys (i.e. 3-nitro-2-pyridine sulfenyl); and for carboxyl groups: methyl ester, tert-butyl ester or benzyl ester. For hydroxyl groups, suitable protecting groups are: methyl, ethyl or tert-butyl; alkoxymethyl or alkoxyethyl; benzyl; acetyl; benzoyl; trityl (Trt) or trialkylsilyl such as tetrabutyldimethylsilyl. For thiol groups, suitable protecting groups are: trityl and 4-methoxybenzyl. The use of further protecting groups are described in ‘Protective Groups in Organic Synthesis’, Theorodora W. Greene and Peter G. M. Wuts, (Third Edition, John Wiley & Sons, 1999).

Preferably, said chemical group capable of reacting with a source of an imaging moiety:
(i) comprises a chelator capable of complexing a metallic imaging moiety;
(ii) comprises an organometallic derivative such as a trialkylstannane or a trialkylsilane;
(iii) comprises a derivative containing an alkyl halide, alkyl tosylate or alkyl mesylate for nucleophilic substitution;
(iv) comprises a derivative containing an aromatic ring activated towards nucleophilic or electrophilic substitution;
(v) comprises a derivative containing a functional group which undergoes facile alkylation; or,
(vi) comprises a derivative which alkylates thiol-containing compounds to give a thioether-containing product.

When the imaging moiety comprises a metal ion, the precursor comprises a chemical group capable of complexing the metal ion to form a metal complex. By the term “metal complex” is meant a coordination complex of the metal ion with one or more ligands. It is strongly preferred that the metal complex is “resistant to transchelation”, i.e. does not readily undergo ligand exchange with other potentially competing ligands for the metal coordination sites. Potentially competing ligands include the LOX binder itself plus other excipients in the preparation in vitro (e.g. radioprotectants or antimicrobial preservatives used in the preparation), or endogenous compounds in vivo (e.g. glutathione, transferrin or plasma proteins).

Suitable ligands for use in the present invention which form metal complexes resistant to transchelation include: chelating agents, where 2-6, preferably 2-4, metal donor atoms are arranged such that 5- or 6-membered chelate rings result (by having a non-coordinating backbone of either carbon atoms or non-coordinating heteroatoms linking the metal donor atoms); or monodentate ligands which comprise donor atoms which bind strongly to the metal ion, such as isonitriles, phosphines or diazenides. Examples of donor atom types which bind well to metals as part of chelating agents are: amines, thiols, amides, oximes, and phosphines. Phosphines form such strong metal complexes that even monodentate or bidentate phosphines form suitable metal complexes. The linear geometry of isonitriles and diazenides is such that they do not lend themselves readily to incorporation into chelating agents, and are hence typically used as monodentate ligands. Examples of suitable isonitriles include simple alkyl isonitriles such as tert-butylisonitrile, and ether-substituted isonitriles such as mibi (i.e. 1-isocyano-2-methoxy-2-methylpropane). Examples of suitable phosphines include Tetrofosmin, and monodentate phosphines such as tris(3-methoxypropyl)phosphine. Examples of suitable diazenides include the HYNIC series of ligands i.e. hydrazine-substituted pyridines or nicotinamides.

Examples of suitable chelating agents for technetium which form metal complexes resistant to transchelation include, but are not limited to:

(i) diaminedioximes;
(ii) N₃S ligands having a thioltriamide donor set such as MAG₃ (mercaptoacetyltriglycine) and related ligands; or having a diamidepyridinethiol donor set such as Pica;
(iii) N₂S₂ ligands having a diaminedithiol donor set such as BAT or ECD (i.e. ethylcysteinate dimer), or an amideaminedithiol donor set such as MAMA;
(iv) N₄ ligands which are open chain or macrocyclic ligands having a tetramine, amidetriamine or diamidediamine donor set, such as cyclam, monoxocyclam or dioxocyclam; or,
(v) N₂O₂ ligands having a diaminediphenol donor set.

Preferred chelating agents of the invention for technetium are diaminedioximes and tetraamines, the preferred versions of which are now described in more detail.

Preferred diaminedioximes are of Formula (X):

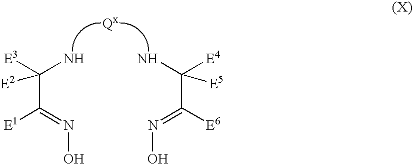

where E¹-E⁶ are each independently an R* group;
each R* is H or C₁₋₁₀ alkyl, C₃₋₁₀ alkylaryl, C₂₋₁₀ alkoxyalkyl, C₁₋₁₀ hydroxyalkyl, C₁₋₁₀ fluoroalkyl, C₂₋₁₀ carboxyalkyl or C₁₋₁₀ aminoalkyl, or two or more R* groups together with the atoms to which they are attached form a carbocyclic, heterocyclic, saturated or unsaturated ring, and wherein one or more of the R* groups is conjugated to the vector;
and Q^{x} is a bridging group of formula -(J¹)_{f}-;
where f is 3, 4 or 5 and each J¹ is independently —O—, —NR*— or —C(R*)₂— provided that -(J¹)_{f}- contains a maximum of one J¹ group which is —O— or —NR*—.

Preferred Q^{x} groups are as follows:

Q^{x}=—(CH₂)(CHR*)(CH₂)— i.e. propyleneamine oxime or PnAO derivatives;
Q^{x}=—(CH₂)₂(CHR*)(CH₂)₂— i.e. pentyleneamine oxime or PentAO derivatives;

### Q^{x}=—(CH₂)₂NR*(CH₂)₂—.

E¹ to E⁶ are preferably chosen from: C₁₋₃ alkyl, alkylaryl alkoxyalkyl, hydroxyalkyl, fluoroalkyl, carboxyalkyl or aminoalkyl. Most preferably, each E¹ to E⁶ group is CH₃.

The LOX binder is preferably conjugated at either the E¹ or E⁶ R* group, or an R* group of the Q^{x} moiety. Most preferably, it is conjugated to an R* group of the Q^{x} moiety. When it is conjugated to an R* group of the Q^{x} moiety, the R* group is preferably at the bridgehead position. In that case, Q^{x} is preferably —(CH₂)(CHR*)(CH₂)—, —(CH₂)₂(CHR*)(CH₂)₂— or —(CH₂)₂NR*(CH₂)₂—, most preferably —(CH₂)₂(CHR*)(CH₂)₂—. An especially preferred bifunctional diaminedioxime chelator has the Formula (Xa):

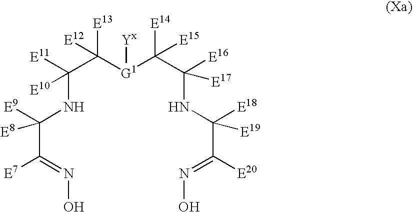

where:
E⁷-E²⁰ are each independently an R* group;

### G¹ is N or CR*;

Y^{x} is -(L⁴)ᵣ-binder, wherein L⁴ and r are as previously defined for L¹ and n, ‘binder’ represents a LOX binder as previously defined. Where -(L⁴)ᵣ- is present there is no other linker connecting the chelate and the LOX binder.

A preferred chelator of Formula (Xa) is of Formula (Xb):

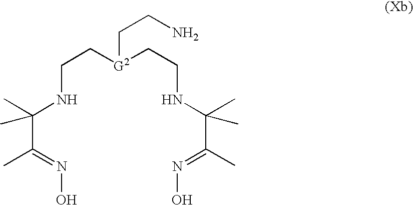

where G² is as defined above for G¹, and is preferably CH (=“chelate X”; synthesis described in Example 6);
such that the LOX binder is conjugated via the bridgehead —CH₂CH₂NH₂ group.

Preferred tetraamine chelators of the invention are of Formula Z:

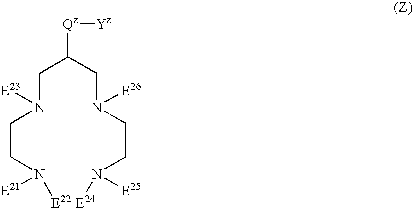

wherein:
Q^{z} is a bridging group of formula -(J²)_{g}-;
where g is 1-8 and each J² is independently —O—, —NR*— or —C(R*)₂—, preferably —C(R*)₂— and most preferably —CH₂—
Y^{z} is -(L⁵)ₛ-binder, wherein L⁵ and s are as previously defined for L¹ and n, but wherein -(L⁵)ₛ- does not contain aryl rings, helping to minimize the lipophilicity of the complex. The term ‘binder’ represents a LOX binder as previously defined. Where -(L⁵)ₛ- is present there are no other linker groups connecting the chelate to the LOX binder.

E²¹ to E²⁶ are an R* group as previously defined.

A most preferred tetraamine chelate of the present invention is of Formula Za:

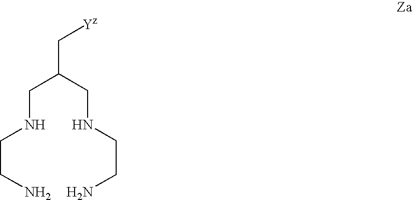

wherein Y^{z} is as defined above.

An especially preferred tetramine chelate of the present invention is of Formula Za wherein Y^{z} is —CO-binder.

The above described ligands are particularly suitable for complexing technetium e.g. ^{94m}Tc or ^{99m}Tc, and are described more fully by Jurisson et al [Chem. Rev., 99, 2205-2218 (1999)]. The ligands are also useful for other metals, such as copper (⁶⁴Cu or ⁶⁷Cu), vanadium (e.g. ⁴⁸V), iron (eg. ⁵²Fe), or cobalt (e.g. ⁵⁵Co). Other suitable ligands are described in Sandoz WO 91/01144, which includes ligands which are particularly suitable for indium, yttrium and gadolinium, especially macrocyclic aminocarboxylate and aminophosphonic acid ligands. Examples of suitable chelating agents with such donor atoms include 1,4,7,10-tetraazacyclododecane-1,4,7,10-tetraacetic acid (DOTA) and diethylenetriaminepentacetic acid (DTPA). Ligands which form non-ionic (i.e. neutral) metal complexes of gadolinium are known and are described in U.S. Pat. No. 4,885,363. When the radiometal ion is technetium, the ligand is preferably a chelating agent which is tetradentate. Preferred chelating agents for technetium are the diaminedioximes, or those having an N₂S₂ or N₃S donor set as described above.

It is envisaged that the role of the linker group [defined above as either -(L⁴)ᵣ- or -(L⁵)ₛ-] is to distance the relatively bulky technetium complex, which results upon metal coordination, from the active site of the LOX binder so that e.g. substrate binding is not impaired. This can be achieved by a combination of flexibility (e.g. simple alkyl chains), so that the bulky group has the freedom to position itself away from the active site and/or rigidity such as a cycloalkyl or aryl spacer which orientates the metal complex away from the active site. The nature of the linker group can also be used to modify the biodistribution of the resulting technetium complex of the conjugate. Thus, e.g. the introduction of ether groups in the linker will help to minimise plasma protein binding, or the use of polymeric linker groups such as polyalkyleneglycol, especially polyethyleneglycol (PEG) can help to prolong the lifetime of the agent in the blood in vivo.

Preferred linker groups -(L⁴)ᵣ- or -(L⁵)ₛ- have a backbone chain (i.e. the linked atoms which make up the -(L⁴)ᵣ- or -(L⁵)ₛ- moiety) which contains 2 to 10 atoms, most preferably 2 to 5 atoms, with 2 or 3 atoms being especially preferred. A minimum linker group backbone chain of 2 atoms confers the advantage that the chelator is well-separated from the biological targeting moiety so that any interaction is minimised. Furthermore, the LOX binder is unlikely to compete effectively with the coordination of the chelator to the metal ion. In this way, both the biological targeting characteristics of the LOX binder, and the metal complexing capability of the chelator is maintained. It is strongly preferred that the LOX binder is bound to the chelator in such a way that the linkage does not undergo facile metabolism in blood. That is because such metabolism would result in the imaging metal complex being cleaved off before the labelled LOX binder reaches the desired in vivo target site. The LOX binder is therefore preferably covalently bound to the metal complexes of the present invention via -(L⁴)ᵣ- or -(L⁵)ₛ- linker groups which are not readily metabolised. Suitable such linkages are carbon-carbon bonds, amide bonds, urea or thiourea linkages, or ether bonds.

Non-peptide linker groups such as alkylene groups or arylene groups have the advantage that there are no significant hydrogen bonding interactions with the conjugated LOX binder so that the linker does not wrap round onto the LOX binder. Preferred alkylene spacer groups are —(CH₂)ₜ— where t is an integer of value 2 to 5. Preferably t is 2 or 3. Preferred arylene spacers are of formula:

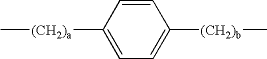

where: a and b are each independently 0, 1 or 2.

A preferred Y group [Y^{x} or Y^{z}] is thus —CH₂CH₂-(L⁶)ᵤ-, —where L⁶ is as defined for L¹ above, and u is an integer of value 0 to 3.

When the LOX binder is a peptide, the Y group is preferably —CH₂CH₂-(L⁷)ᵥ- where -(L⁷)ᵥ- is —CO— or —NR′—, with R′ as previously defined. For Formula Xb, when G² is N, this grouping has the additional advantage that it stems from the symmetrical intermediate N(CH₂CH₂NH₂)₃, which is commercially available.

When the imaging metal is technetium, the usual technetium starting material is pertechnetate, i.e. TcO₄— which is technetium in the Tc(VII) oxidation state. Pertechnetate itself does not readily form metal complexes, hence the preparation of technetium complexes usually requires the addition of a suitable reducing agent such as stannous ion to facilitate complexation by reducing the oxidation state of the technetium to the lower oxidation states, usually Tc(I) to Tc(V). The solvent may be organic or aqueous, or mixtures thereof. When the solvent comprises an organic solvent, the organic solvent is preferably a biocompatible solvent, such as ethanol or DMSO. Preferably the solvent is aqueous, and is most preferably isotonic saline.

Where the imaging moiety is radioiodine, preferred precursors are those which comprise a derivative which either undergoes electrophilic or nucleophilic iodination or undergoes condensation with a labelled aldehyde or ketone. Examples of the first category are:
(a) organometallic derivatives such as a trialkylstannane (eg. trimethylstannyl or tributylstannyl), or a trialkylsilone leg. trimethylsilyl) or an organoboron compound leg. boronate esters or organotrifluoroborates);
(b) a non-radioactive alkyl bromide for halogen exchange or alkyl tosylate, mesylate or triflate for nucleophilic iodination;
(c) aromatic rings activated towards electrophilic iodination (eg. phenols) and aromatic rings activated towards nucleophilic iodination (eg. aryl iodonium salt aryl diazonium, aryl trialkylammonium salts or nitroaryl derivatives).

The precursor preferably comprises: a non-radioactive halogen atom such as an aryl iodide or bromide (to permit radioiodine exchange); an activated precursor aryl ring (e.g. a phenol group); an organometallic precursor compound (e.g. trialkyltin, trialkylsilyl or organoboron compound); or an organic precursor such as triazenes or a good leaving group for nucleophilic substitution such as an iodonium salt. Preferably for radioiodination, the precursor comprises an organometallic precursor compound, most preferably trialkyltin.

Precursors and methods of introducing radioiodine into organic molecules are described by Bolton [J. Lab. Comp. Radiopharm., 45, 485-528 (2002)]. Suitable boronate ester organoboron compounds and their preparation are described by Kabalaka et al [Nucl. Med. Biol., 29, 841-843 (2002) and 30, 369-373 (2003)]. Suitable organotrifluoroborates and their preparation are described by Kabalaka et al [Nucl. Med. Biol., 31, 935-938 (2004)].

Examples of aryl groups to which radioactive iodine can be attached are given below:

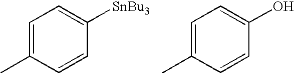

Both contain substituents which permit facile radioiodine substitution onto the aromatic ring. Alternative substituents containing radioactive iodine can be synthesised by direct iodination via radiohologen exchange, e.g.

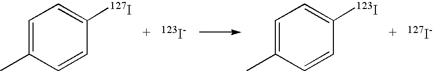

The radioiodine atom is preferably attached via a direct covalent bond to an aromatic ring such as a benzene ring, or a vinyl group since it is known that iodine atoms bound to saturated aliphatic systems are prone to in vivo metabolism and hence loss of the radioiodine.

When the imaging moiety is a radioactive isotope of fluorine the radiofluorine atom may form part of a fluoroalkyl or fluoroalkoxy group, since alkyl fluorides are resistant to in vivo metabolism. Alternatively, the radiofluorine atom may be attached via a direct covalent bond to an aromatic ring such as a benzene ring. Radiohalogenation may be carried out via direct labelling using the reaction of ¹⁸F-fluoride with a suitable chemical group in the precursor having a good leaving group, such as an alkyl bromide, alkyl mesylate or alkyl tosylate. ¹⁸F can also be introduced by alkylation of N-haloacetyl groups with a ¹⁸F(CH₂)₃OH reactant, to give —NH(CO)CH₂O—(CH₂)₃¹⁸F derivatives. For aryl systems, ¹⁸F-fluoride nucleophilic displacement from an aryl diazonium salt, aryl nitro compound or an aryl quaternary ammonium salt are suitable routes to aryl-¹⁸F derivatives.

A further approach for radiofluorination as described in WO 03/080544, is to react a precursor compound comprising one of the following substituents:

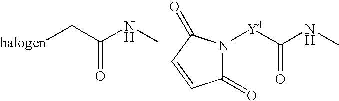

with a compound of Formula VI:

¹⁸F—Y⁵—SH (VI)

wherein Y⁴ is a linker of formula -(L⁸)_{w}- wherein L⁸ is as previously defined for L¹, w is 1-10 and optionally includes 1-6 heteroatoms; and,
Y⁵ is a linker of formula -(L⁹)ₜ- wherein L⁹ is as previously defined for L¹, x is 1-30 and optionally includes 1 to 10 heteroatoms;
to give radiofluorinated imaging agents of formula (VIa) or (VIb) respectively:

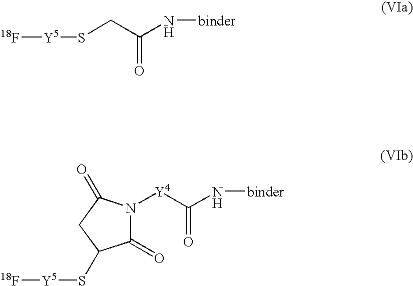

wherein Y⁴ and Y⁵ are as defined above, and ‘binder’ is a LOX binder, as described above in relation to the imaging agent of the invention.

Further details of synthetic routes to ¹⁸F-labelled derivatives are described by Bolton, J. Lab. Comp. Radiopharm., 45, 485-528 (2002).

A ¹⁸F-labelled compound of the invention may be obtained by formation of ¹⁸F fluorodialkylamines and subsequent amide formation when the ¹⁸F fluorodialkylamine is reacted with a precursor containing, e.g. chlorine, P(O)Ph₃ or an activated ester.

The following table illustrates some examples of precursors of the present invention:

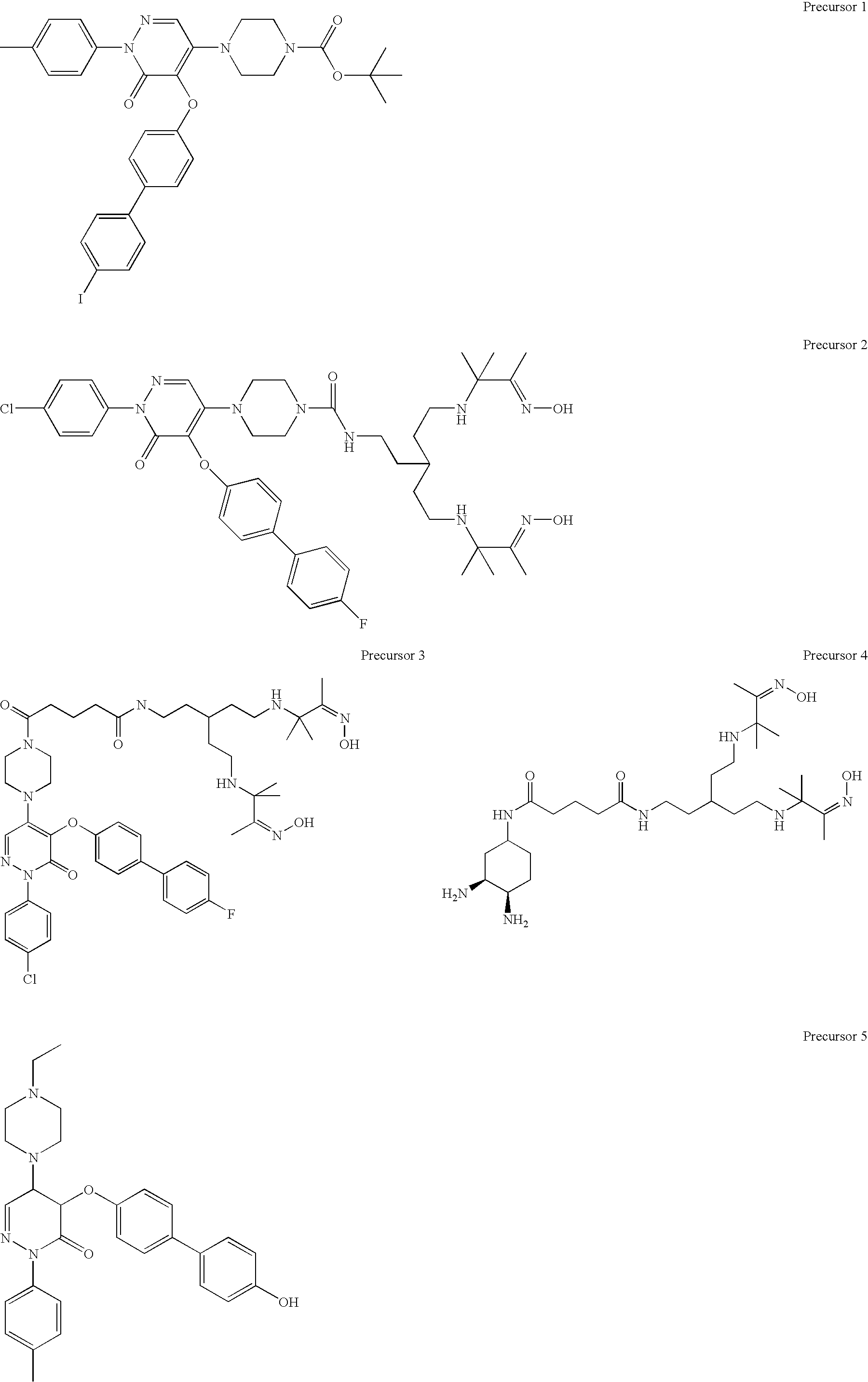

Precursor 1 is suitable for radioiodination by iodine exchange with ¹²³I to form Imaging agent 6. Precursors 2, 3 and 4 are suitable for complexation with ^{99m}Tc to form Imaging agents 7, 8 and 13. Precursor 5 is suitable for radioiodine substitution onto the phenol to form another imaging agent.

Methods for the synthesis of Precursors 1, 3, 4 and 5 are described in Examples 2, 8, 10 and 12.

A further aspect of the present invention is a precursor as defined in relation to the method of preparation of the imaging agent, wherein said chemical group:
(i) comprises a chelator capable of complexing a metallic imaging moiety;
(ii) comprises an organometallic derivative such as a trialkylstannane or a trialkylsilane;
(iii) comprises a derivative containing an alkyl halide, alkyl tosylate or alkyl mesylate for nucleophilic substitution; or,
(iv) comprises a derivative which alkylates thiol-containing compounds to give a thioether-containing product.

In another further aspect, the present invention provides a pharmaceutical composition comprising the imaging agent as described above, together with a biocompatible carrier, in a form suitable for mammalian administration. In a preferred embodiment, the pharmaceutical composition is a radiopharmaceutical composition.

The “biocompatible carrier” is a fluid, especially a liquid, in which the imaging agent is suspended or dissolved, such that the composition is physiologically tolerable, i.e. can be administered to the mammalian body without toxicity or undue discomfort. The biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). The biocompatible carrier medium may also comprise biocompatible organic solvents such as ethanol. Such organic solvents are useful to solubilise more lipophilic compounds or formulations. Preferably the biocompatible carrier medium is pyrogen-free water for injection, isotonic saline or an aqueous ethanol solution. The pH of the biocompatible carrier medium for intravenous injection is suitably in the range 4.0 to 10.5.

Such pharmaceutical compositions are suitably supplied in either a container which is provided with a seal which is suitable for single or multiple puncturing with a hypodermic needle (e.g. a crimped-on septum seal closure) whilst maintaining sterile integrity. Such containers may contain single or multiple patient doses. Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. Where the pharmaceutical composition is a radiopharmaceutical composition, the pre-filled syringe may optionally be provided with a syringe shield to protect the operator from radioactive dose. Suitable such radiopharmaceutical syringe shields are known in the art and preferably comprise either lead or tungsten.

The pharmaceuticals of the present invention may be prepared from kits, as is described below in an additional aspect of the invention. Alternatively, they may be prepared under aseptic manufacture conditions to give the desired sterile product. The pharmaceuticals may also be prepared under non-sterile conditions, followed by terminal sterilisation using e.g. gamma-irradiation, autoclaving, dry heat or chemical treatment (e.g. with ethylene oxide). Preferably, the pharmaceuticals of the present invention are prepared from kits.

As described above in relation to the imaging agent of the invention, for radiopharmaceutical compositions, the most preferred radioactive imaging moieties of the invention are ^{99m}Tc, ¹²³I, ¹¹C and ¹⁸F.

In an additional aspect, the present invention provides kits for the preparation of the pharmaceutical compositions of the invention. Such kits comprise a suitable precursor of the invention, preferably in sterile non-pyrogenic form, so that reaction with a sterile source of an imaging moiety gives the desired pharmaceutical with the minimum number of manipulations. Such considerations are particularly important in the case of radiopharmaceuticals, in particular for radiopharmaceuticals where the radioisotope has a relatively short half-life, for ease of handling and hence reduced radiation dose for the radiophormacist. Hence, the reaction medium for reconstitution of such kits is preferably a “biocompatible carrier” as defined above, and is most preferably aqueous.

Suitable kit containers comprise a sealed container which permits maintenance of sterile integrity and/or radioactive safety, plus optionally an inert headspace gas (e.g. nitrogen or argon), whilst permitting addition and withdrawal of solutions by syringe. A preferred such container is a septum-sealed vial, wherein the gas-tight closure is crimped on with an overseal (typically of aluminium). Such containers have the additional advantage that the closure can withstand vacuum if desired e.g. to change the headspace gas or degas solutions.

Preferred aspects of the precursor when employed in the kit are as described above in relation to the method of synthesis. The precursors for use in the kit may be employed under aseptic manufacture conditions to give the desired sterile, non-pyrogenic material. The precursors may also be employed under non-sterile conditions, followed by terminal sterilisation using e.g. gamma-irradiation, autoclaving, dry heat or chemical treatment (e.g. with ethylene oxide). Preferably, the precursors are employed in sterile, non-pyrogenic form. Most preferably the sterile, non-pyrogenic precursors are employed in the sealed container as described above.

The precursor of the kit is preferably supplied covalently attached to a solid support matrix as described above in relation to the method of synthesis.

For ^{99m}Tc, the kit is preferably lyophilised and is designed to be reconstituted with sterile ^{99m}Tc-pertechnetate (TcO₄⁻) from a ^{99m}Tc radioisotope generator to give a solution suitable for human administration without further manipulation. Suitable kits comprise a container (e.g. a septum-sealed vial) containing the uncomplexed chelating agent, together with a pharmaceutically acceptable reducing agent such as sodium dithionite, sodium bisulphite, ascorbic acid, formamidine sulphinic acid, stannous ion, Fe(II) or Cu(I); together with at least one salt of a weak organic acid with a biocompatible cation. By the term “biocompatible cation” is meant a positively charged counterion which forms a salt with an ionised, negatively charged group, where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred biocompatible cations are sodium and potassium, most preferably sodium.

The kits for preparation of ^{99m}Tc imaging agents may optionally further comprise a second, weak organic acid or salt thereof with a biocompatible cation, which functions as a transchelator. The transchelator is a compound which reacts rapidly to form a weak complex with technetium, then is displaced by the chelator of the kit. This minimises the risk of formation of reduced hydrolysed technetium (RHT) due to rapid reduction of pertechnetate competing with technetium complexation. Suitable such transchelators are the weak organic acids and salts thereof described above, preferably tartrates, gluconates, glucoheptonates, benzoates, or phosphonates, preferably phosphonates, most especially diphosphonates. A preferred such transchelator is MDP, ie. methylenediphosphonic acid, or a salt thereof with a biocompatible cation.

Also in relation to ^{99m}Tc kits, an alternative to use of the chelator in free form, the kit may optionally contain a non-radioactive metal complex of the chelator which, upon addition of the technetium, undergoes transmetallation (i.e. ligand exchange) giving the desired product. Suitable such complexes for transmetallation are copper or zinc complexes.

The pharmaceutically acceptable reducing agent used in the ^{99m}Tc imaging agent kit is preferably a stannous salt such as stannous chloride, stannous fluoride or stannous tartrate, and may be in either anhydrous or hydrated form. The stannous salt is preferably stannous chloride or stannous fluoride.

The kits may optionally further comprise additional components such as a radioprotectant, antimicrobial preservative, pH-adjusting agent or filler.

By the term “radioprotectant” is meant a compound which inhibits degradation reactions, such as redox processes, by trapping highly-reactive free radicals, such as oxygen-containing free radicals arising from the radiolysis of water. The radioprotectants of the present invention are suitably chosen from: ascorbic acid, poro-aminobenzoic acid (i.e. 4-aminobenzoic acid), gentisic acid (i.e. 2,5-dihydroxybenzoic acid) and salts thereof with a biocompatible cation. The “biocompatible cation” and preferred embodiments thereof are as described above.

By the term “antimicrobial preservative” is meant an agent which inhibits the growth of potentially harmful micro-organisms such as bacteria, yeasts or moulds. The antimicrobial preservative may also exhibit some bactericidal properties, depending on the dose. The main role of the antimicrobial preservatives) of the present invention is to inhibit the growth of any such micro-organism in the radiopharmaceutical composition post-reconstitution, i.e. in the radioactive diagnostic product itself. The antimicrobial preservative may, however, also optionally be used to inhibit the growth of potentially harmful micro-organisms in one or more components of the non-radioactive kit of the present invention prior to reconstitution. Suitable antimicrobial preservative(s) include: the parabens, i.e. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the reconstituted kit is within acceptable limits (approximately pH 4.0 to 10.5) for human or mammalian administration. Suitable such pH-adjusting agents include pharmaceutically acceptable buffers, such as tricine, phosphate or TRIS [ie. tris(hydroxymethyl)aminomethane], and pharmaceutically acceptable bases such as sodium carbonate, sodium bicarbonate or mixtures thereof. When the precursor is employed in acid salt form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

The imaging agent of the invention is useful for in vivo imaging. Accordingly, in a yet further aspect, the present invention provides on imaging agent of the invention for use in an in vivo diagnostic or imaging method, e.g. SPECT or PET. Preferably said method relates to the in vivo imaging of a condition in which LOX is upregulated and therefore has utility in the diagnosis of conditions associated with fibrosis such as liver fibrosis, congestive heart failure, glomerulosclerosis and respiratory failure. In a most preferred embodiment, said condition is liver fibrosis.

This aspect of the invention also provides a method for the in vivo diagnosis or imaging in a subject of a condition in which LOX is upregulated, comprising prior administration of the pharmaceutical composition of the invention. Said subject is preferably a mammal and most preferably a human. In an alternative embodiment, this aspect of the invention furthermore provides for the use of the imaging agent of the invention for imaging in vivo of a condition in which LOX is upregulated in a subject wherein said subject is previously administered with the pharmaceutical composition of the invention.

By “previously administered” is meant that the step involving the clinician, wherein the pharmaceutical is given to the patient e.g., intravenous injection, has already been carried out. This aspect of the invention also encompasses use of the imaging agent of the invention for the manufacture of pharmaceutical for the diagnostic imaging in vivo of a condition in which LOX is upregulated.

In an even further aspect the invention provides a method of monitoring the effect of treatment of a human or animal body with a drug to combat a condition in which LOX is upregulated, said method comprising administering to said body an imaging agent of the invention and detecting the uptake of said imaging agent, said administration and detection optionally but preferably being effected repeatedly, e.g. before, during and after treatment with said drug.

### Brief Description of the Examples

Example 1 describes the synthesis of a pyridazinone LOX binder.

Example 2 describes the synthesis of a pyridazinone-based precursor compound suitable for radioiodination (“precursor 1”).

Example 3 describes the synthesis of a homocysteine lactone.

Examples 4 and 5 describe the synthesis non-radioactive versions of imaging agents 3 and 4.

Example 6 describes the synthesis of chelate X.

Example 7 describes the synthesis of the glutarylamide derivative of chelate X.

Example 8 describes the synthesis of Precursor 3, suitable for labelling with ^{99m}Tc to form Imaging Agent 8.

Example 9 describes how to label Precursor 3 with ^{99m}Tc to form Imaging Agent 8.

Example 10 describes the synthesis of ^{99m}Tc-labelled Imaging Agent 13.

Example 11 describes the synthesis of non-radioactive Imaging Agent 9.

Example 12 describes the synthesis of Precursor 5.

### EXAMPLES

List of abbreviations used in Examples

| | |
|---|---|
| Boc | t-butoxycarbonyl |
| DMF | dimethyl formamide |
| ESI-MS | electrospray ionisation mass spectrometry |
| HATU | O-(7-Azabenzotriazole-1-yl)-N,N,N′N′- |
| | tetramethyluronium hexafluorophosphate |
| LC-MS | liquid chromatography mass spectrometry |
| Lys | lysine |
| NMM | N-Methylmorpholine |
| PyAOP | (7-azabenzotriazole-1-yloxy) tripyrrolidino- |
| | phosphonium hexafluorophosphate |
| TFA | trifluoroacetic acid. |

### Example 1

### Synthesis of 4-(5-Chloro-6-oxo-1-p-tolyl-1,6-dihydro-4-pyridazinyl)-piperazine-1-carboxylic acid tert-butyl ester

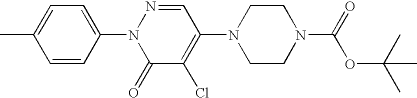

Boc-piperazine (Acros, 0.373 g, 2.0 mmol) was added slowly to a solution of 4,5-dichloro-2-(4-methylphenyl)-2,3-dihydropyridazin-3-one (Maybridge, 0.255 g, 1.0 mmol) in dichloromethane (10 ml) and refluxed for 20 hours. The solution was washed with sodium hydroxide (1 M), dried (Na₂SO₄) and concentrated. The product was purified by column chromatography (silica, dichloromethane/methanol 99:1) giving 0.236 g, 58% yield. LC-MS analysis (column Phenomenex Luna C18(2) 3 μm 2.0×50 mm, solvents: A=water/0.1% TFA and B=acetonitrile/0.1% TFA; gradient 10-80% B over 10 min; flow 0.3 ml/min, UV detection at 214 and 254 nm, ESI-MS; t_{R}=8.4 min, m/z 405.1 (MH+)) confirmed the structure.

### Example 2

### Synthesis of 4-[5-(4′-Iodo-biphenyl-4-yloxy)-6-oxo-1-p-tolyl-1,6-dihydro-4-pyridazinyl]-piperazine-1-carboxylic acid tert-butyl ester [precursor 1]

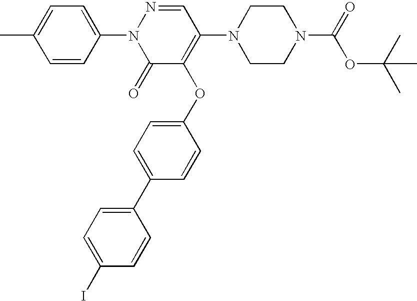

To a suspension of Cs₂CO₃ (Fluka, 0.164 g, 0.50 mmol) in dry methanol (1 ml) was added 4-hydroxy-4′-iodobiphenyl (Alfa Aesar, 0.299 g, 1.0 mmol) in portions. The reaction was run under argon. The mixture was stirred for one hour and concentrated. To the residue was added a solution from a) above (0.136 g, 0.336 mmol) in DMF (3.5 ml). The mixture was stirred at 120° C. for 20 h. Analysis by LC-MS (column Phenomenex Luna C18(2) 3 μm 2.0×50 mm, solvents: A=water/0.1% TFA and B=acetonitrile/0.1% TFA; gradient 50-95% B over 10 min; flow 0.3 ml/min, UV detection at 214 and 254 nm, ESI-MS; t_{R}=8.0 min, m/z 665.2 (MH+)) confirmed the product.

### Example 3

### Synthesis of lysine-homocysteine thiolactone [H-Lys-Hcv-thiolactone]

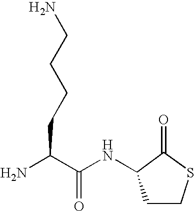

Boc-Lys(Boc)-OSu (44 mg), N-methylmorpholine (NMM) (44 μL) and L-Homocysteine thiolactone HCl salt (15 mg) were dissolved in 1 mL dimethylformamide (DMF) and the reaction mixture stirred for 16 hours. DMF was evaporated in vacuo and the residue treated with 10 mL trifluoroacetic acid (TFA) containing 5% water for 30 minutes. TFA was evaporated in vacuo and preparative HPLC lisocratic: 1000% H₂O/0.1% TFA over 40 min, flow rate: 10 mL/min, column: Phenomenex Luna 5μ C18 (2) 250×21.20 mm, detection: UV 214 nm, product retention time: 14.7 min) of the residue afforded 36 mg pure product. The pure product was analysed by analytical HPLC (isocratic: 100% H₂O/0.1% TFA over 10 min, flow rate: 0.3 mL/min, column: Phenomenex Luna 3μ C18 (2) 50×2 mm, detection: UV 214 nm, product retention time: 1.06 min). Further product characterisation was carried out using mass spectrometry (MH⁺ calculated: 246.1, MH⁺ found: 246.1).

### Example 4

### Synthesis of non-radioactive Imaging agent 3 [H-Lys(3-(4-hydroxy-3-iodophenyl)propionyl)-Hcy-thiolactone]

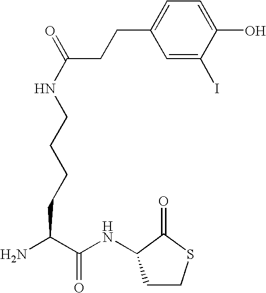

Boc-Lys-OH (25 mg), N-succinimidyl 3-(4-hydroxy-3-iodophenyl)propionate (19 mg) and NMM (22 μL) were dissolved in DMF (1 mL) and the mixture stirred for 3 days. Purification using preparative HPLC afforded 26 mg pure Boc-Lys(3-(4-hydroxy-3-iodophenyl) propionyl)-OH. Boc-Lys(3-(4-hydroxy-3-iodophenyl) propionyl)-OH (26 mg), NMM (22 μL) and L-Homocysteine thiolactone HCl salt (15 mg) were dissolved in DMF (1 mL). (7-Azabenzotriazole-1-yloxy)tripyrrolidinophosphonium hexafluorophosphate (PyAOP) (26 mg) was added and the reaction mixture stirred for 80 minutes. DMF was evaporated in vacuo and the residue treated with TFA (10 mL) containing 5% water for 30 minutes. TFA was evaporated in vacuo and preparative HPLC (gradient: 10-40% B over 40 min where A=H₂O/0.1% TFA and B=ACN/0.1% TFA, flow rate: 10 mL/min, column: Phenomenex Luna 5μ C18 (2) 250×21.20 mm, detection: UV 214 nm, product retention time: 30 min) of the residue afforded 22 mg pure product. The pure product was analysed by analytical HPLC (gradient: 10-40% B over 10 min where A=H₂O/0.1% TFA and B=ACN/0.1% TFA, flow rate: 0.3 mL/min, column: Phenomenex Luna 3μ C18 (2) 50×2 mm, detection: UV 214 nm, product retention time: 5.28 min). Further product characterisation was carried out using mass spectrometry (MH⁺ calculated: 520.1, MH⁺ found: 520.21.

### Example 5

### Synthesis of non-radioactive Imaging agent 4 [3-(4-hydroxy-3-iodophenyl)propionyl-Lys-Hcy-thiolactone]

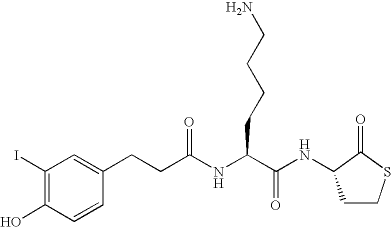

H-Lys(Boc)-OH (25 mg), N-succinimidyl 3-(4-hydroxy-3-iodophenyl)propionate (19 mg) and NMM (22 μL) were dissolved in DMF (1 mL) and the mixture stirred for 3 days. Purification using preparative HPLC afforded 26 mg pure 3-(4-hydroxy-3-iodophenyl)propionyl-Lys(Boc)-OH 3-(4-hydroxy-3-iodophenyl)propionyl-Lys(Boc)-OH (26 mg), NMM (22 μL) and L-Homocysteine thiolactone HCl salt (15 mg) were dissolved in DMF (1 mL). (PyAOP) (26 mg) was added and the reaction mixture stirred for 60 minutes, DMF was evaporated in vacuo and the residue treated with TFA (10 mL) containing 5% water for 30 minutes. TFA was evaporated in vacuo and preparative HPLC (gradient: 10-40% B over 40 min where A=H₂O/0.1% TFA and B=ACN/0.1% TFA, flow rate: 10 mL/min, column: Phenomenex Luna 5μ C18 (2) 250×21.20 mm, detection: UV 214 nm, product retention time: 26 min) of the residue afforded 22 mg pure product. The pure product was analysed by analytical HPLC (gradient: 10-40% B over 5 min where A=H₂O/0.1% TFA and B=ACN/0.1% TFA, flow rate: 0.6 mL/min, column: Phenomenex Luna 3μ C18 (2) 20×2 mm, detection: UV 214 nm, product retention time: 2.62 min). Further product characterisation was carried out using mass spectrometry (MH⁺ calculated: 520.1, MH⁺ found: 520.1).

### Example 6

### Synthesis of chelate X [bis[N-(1,1-dimethyl-2-N-hydroxyimine propyl)-2-aminoethyl]-(2-aminoethyl)methane]

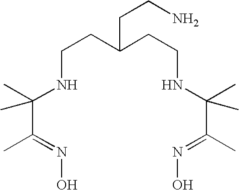

### (Step a): Preparation of tris(methyloxycarbonylmethyl)methane

3-(methoxycarbonylmethylene)glutaric acid dimethylester (89 g, 267 mmol) in methanol (200 ml) was shaken with (10% palladium on charcoal: 50% water) (9 g) under an atmosphere of hydrogen gas (3.5 bar) for (30 h). The solution was filtered through kieselguhr and concentrated in vacuo to give 3-(methoxycarbonylmethyl)glutaric acid dimethylester as an oil, yield (84.9 g, 94%).

NMR ¹H(CDCl₃), δ 2.48 (6H, d, J=8 Hz, 3×CH₂), 2.78 (1H, hextet, J=8 Hz CH,) 3.7 (9H s, 3×CH₃).

NMR ¹³C(CDCl₃), δ 28.6, CH; 37.50, 3×CH₃; 51.6, 3×CH₂; 172.28, 3×COO.

### (Step b): Amidation of trimethylester with p-methoxy-benzylamine

Tris(methyloxycorbonylmethyl)methane [2 g, 8.4 mmol] was dissolved in p-methoxy-benzylamine (25 g, 178.6 mmol). The apparatus was set up for distillation and heated to 120° C. for 24 hrs under nitrogen flow. The progress of the reaction was monitored by the amount of methanol collected. The reaction mixture was cooled to ambient temperature and 30 ml of ethyl acetate was added, then the precipitated-triamide product stirred for 30 min. The triamide was isolated by filtration and the filter cake washed several times with sufficient amounts of ethyl acetate to remove excess p-methoxy-benzylamine. After drying 4.6 g, 100%, of a white powder was obtained. The highly insoluble product was used directly in the next step without further purification or characterisation.

### (Step c): Preparation of 1,1,1-tris[2-(p-methoxybenzylomino)ethyl]methane

To a 1000 ml 3-necked round bottomed flask cooled in a ice-water bath the triamide from step 2(a) (10 g, 17.89 mmol) is carefully added to 250 ml of 1M borane solution (3.5 g, 244.3 mmol) borane. After complete addition the ice-water bath is removed and the reaction mixture slowly heated to 60° C. The reaction mixture is stirred at 60° C. for 20 hrs. A sample of the reaction mixture (1 ml) was withdrawn, and mixed with 0.5 ml 5N HCl and left standing for 30 min. To the sample 0.5 ml of 50 NaOH was added, followed by 2 ml of water and the solution was stirred until all of the white precipitate dissolved. The solution was extracted with ether (5 ml) and evaporated. The residue was dissolved in acetonitrile at a concentration of 1 mg/ml and analysed by MS. If mono- and diamide (M+H/z=520 and 534) are seen in the MS spectrum, the reaction is not complete. To complete the reaction, a further 100 ml of 1M borane THF solution is added and the reaction mixture stirred for 6 more hrs at 60° C. and a new sample withdrawn following the previous sampling procedure. Further addition of the 1M borane in THF solution is continued as necessary until there is complete conversion to the triamine.

The reaction mixture is cooled to ambient temperature and 5N HCl is slowly added, [CARE: vigorous foam formation occurs!]. HCl was added until no more gas evolution is observed. The mixture was stirred for 30 min and then evaporated. The cake was suspended in aqueous NaOH solution 120-40%; 1:2 w/v) and stirred for 30 minutes. The mixture was then diluted with water (3 volumes). The mixture was then extracted with diethylether (2×150 ml) [CARE: do not use halogenated solvents]. The combined organic phases were then washed with water (1×200 ml), brine (150 ml) and dried over magnesium sulphate. Yield after evaporation: 7.6 g, 84% as oil.

NMR ¹H(CDCl₃), δ: 1.45, (6H, m, 3×CH₂; 1.54, (1H, septet, CH); 2.60 (6H, t, 3×CH₂N); 3.68 (6H, s, ArCH₂); 3.78 (9H, s, 3×CH₃O); 6.94 (6H, d, 6×Ar). 7.20 (6H, d, 6×Ar).

NMR ¹³C(CDCl₃), δ: 32.17, CH; 34.44, CH₂; 47.00, CH₂; 53.56, ArCH₂; 55.25, CH₃O; 113.78, Ar; 129.29, Ar; 132.61; Ar; 158.60, Ar.

### (Step d): Preparation of 1,1,1-tris(2-aminoethyl)methane

1,1,1-tris[2-(p-methoxybenzylamino)ethyl]methane (20.0 gram, 0.036 mol) was dissolved in methanol (100 ml) and Pd(OH)₂ (5.0 gram) was added. The mixture was hydrogenated (3 bar, 100° C., in an autoclave) and stirred for 5 hours. Pd(OH)₂ was added in two more portions (2×5 gram) after 10 and 15 hours respectively.

The reaction mixture was filtered and the filtrate was washed with methanol. The combined organic phase was evaporated and the residue was distilled under vacuum (1×10⁻², 110° C.) to give 2.60 gram (50%) of 1,1,1-tris(2-aminoethyl)methane. NMR ¹H(CDCl₃), δ 2.72 (6H, t, 3×CH₂N), 1.41 (H, septet, CH), 1.39 (6H, q, 3×CH₂). NMR ¹³C(CDCl₃), δ 39.8 (CH₂NH₂), 38.2 (CH₂), 31.0 (CH).

### (Step e): Preparation of Chelate X

To a solution of tris(2-aminoethyl)methane (4.047 g, 27.9 mmol) in dry ethanol (30 ml) was added potassium carbonate anhydrous (7.7 g, 55.8 mmol, 2 eq) at room temperature with vigorous stirring under a nitrogen atmosphere. A solution of 3-chloro-3-methyl-2-nitrosobutane (7.56 g, 55.8 mol, 2 eq) was dissolved in dry ethanol (100 ml) and 75 ml of this solution was dripped slowly into the reaction mixture. The reaction was followed by TLC on silica [plates run in dichloromethane, methanol, concentrated (0.88 sg) ammonia; 100/30/5 and the TLC plate developed by spraying with ninhydrin and heating]. The mono-, di- and tri-alkylated products were seen with RF's increasing in that order. Analytical HPLC was run using RPR reverse phase column in a gradient of 7.5-75% acetonitrile in 3% aqueous ammonia. The reaction was concentrated in vacuo to remove the ethanol and resuspended in water (110 ml). The aqueous slurry was extracted with ether (100 ml) to remove some of the trialkylated compound and lipophilic impurities leaving the mono and desired dialkylated product in the water layer. The aqueous solution was buffered with ammonium acetate (2 eq, 4.3 g, 55.8 mmol) to ensure good chromatography. The aqueous solution was stored at 4° C. overnight before purifying by automated preparative HPLC.

Yield (2.2 g, 6.4 mmol, 23%).

Mass spec; Positive ion 10 V cone voltage. Found: 344; calculated M+H=344.

NMR ¹H(CDCl₃), δ 1.24 (6H, s, 2×CH₃), 1.3 (6H, s, 2×CH₃), 1.25-1.75 (7H, m, 3×CH₂, CH), (3H, s, 2×CH₂), 2.58 (4H, m, CH₂N), 2.88 (2H, t CH₂N₂), 5.0 (6H, s, NH₂, 2×NH, 2×OH).

NMR ¹H((CD₃)₂SO) δ1.1 4×CH; 1.29, 3×CH₂; 2.1 (4H, t, 2×CH₂);

NMR ¹³C((CD₃)₂SO), δ 9.0 (4×CH₃), 25.8 (2×CH₃), 31.0 2×CH₂, 34.6 CH₂, 56.8 2×CH₂N, 160.3; C═N.

HPLC conditions: flow rate 8 ml/min using a 25 mm PRP column
A = 3% ammonia solution (sp. gr = 0.88)/water; B = Acetonitrile

| Time | % B |
|---|---|
| 0 | 7.5 |
| 15 | 75.0 |
| 20 | 75.0 |
| 22 | 7.5 |
| 30 | 7.5 |

Load 3 ml of aqueous solution per run, and collect in a time window of 12.5-13.5 min.

### Example 7

### Synthesis of the glutarylamide derivative of chelate X [bis[(1,1-dimethyl-2-N-hydroxyimine propyl)2-aminoethyl]-(2-(Glutarylamide)ethyl)methane]

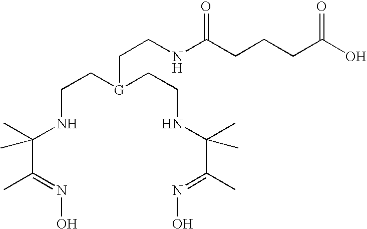

Chelate X (0.5 g, 1.45 mmol) in dry acetonitrile (50 ml) and triethylamine (150 mg, 1.45 mmol) under an atmosphere of nitrogen was cooled on an ice bath to 0° C. Glutaric anhydride (165 mg, 1.45 mmol) was added to the stirred reaction and allowed to warm to room temperature and left to stir overnight. The precipitate that formed overnight was collected by filtration and dried in vacuo to give on impure sample of the title compound (267 mg, 0.583 mmol, 40%). The filtrate was concentrated in vacuo to give a colourless glass which together with the precipitate that had been collected was redissolved in 5% 0.880 sg ammonia, water (50 ml) and purified by automated preparative HPLC.

HPLC conditions: flow rate 8 ml/min, using a 150 mm×25 mm PRP column

Sample loaded in 2 ml of solution per run.

A = 3% ammonia solution (sp. gr = 0.88)/water.
B = Acetonitrile

| Time | % B |
|---|---|
| 0 | 7.5 |
| 15 | 75.0 |
| 20 | 75.0 |
| 22 | 7.5 |
| 31 | 7.5 |

The required product eluted at 15.25-16.5 min. The product solution was evaporated in vacuo to give (304 mg, 0.68 mmol, 47%) of a colourless glassy foam m.p. 54.8° C. The product analysed as one spot on both TLC and analytical HPLC.

NMR ¹H(DMSO), 0.7 (12H, s, 4×CH₃), 0.85 (4H, m, 2×CH₂), 1.0 (1H, m, CH), 1.3 (6H, s, 2×CH₃), 1.3 (4H, m, 2×CH₂), 1.6 (2H, m, CH₂), 1.75 (6, m, 3×CH₂), 2.6 (2, m, 2×OH) 3.2 (2H, t, NH) 7.3 (1H, t, NH).

NMR ¹³C(CD₃SO) 8.97, 20.51, 20.91, 25.09, 25.60, 31.06, 33.41, 33.86, 56.89, 66.99 160.07, 1712.34, 174.35 174.56

M/S C₂₂H₄₃N₅O₅ M+H=457 Found 457.6

### Example 8

### Synthesis of Precursor 3 [5-{4-[1-(4-chloro-phenyl)-5-(4′-fluoro-biphenyl-4-yloxy)-6-oxo-1,6-dihydro-pyridazin-4-yl]-piperazin-1-yl}-5-oxopentanoic acid {5-(2-hydroxyimino-1,1-dimethyl-propylamino)-3-[2-(2-hydroxyimino-1,1-dimethyl-propylamino)ethyl]pentyl}amide]

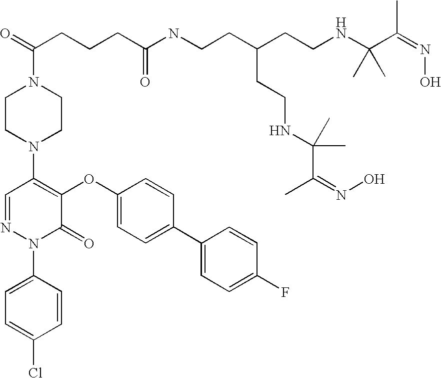

To the glutarylamide derivative of chelate X (300 mg, 0.66 mmol) in DMF (2 mL) was added HATU (249 mg, 0.66 mmol) and NMM (132 μL, 1.32 mmol). The mixture was stirred for 5 minutes and tetrafluorothiophenol (0.66 mmol, 119 mg) was added. After stirring for 10 minutes the reaction mixture was diluted with 20% acetonitrile/water (8 mL) and the product was purified by RP-HPLC to yield 110 mg of the desired product following freeze-drying.

To a solution of 2-(4-chloro-phenyl)-4-(4′-fluorobiphenyl-4-yloxy)-5-piperazin-1-yl-2H-pyridazin-3-one (0.5 mmol, synthesised by the methods described in WO 03/097612) in DMF (5 ml) is added N-methylmorpholine (1 mmol) and the pentafluorophenyl ester described above (0.55 mmol). The reaction mixture is stirred for 1 hr and concentrated in vacuo. The residue is taken up in a mixture of water and acetonitrile containing 0.1% TFA and purified by reverse phase chromatography using a suitable water/acetonitrile (0.1% TFA) gradient.

### Example 9

### ^{99m}Tc Labeling of Precursor 3 to Form Imaging Agent 8 [Prophetic Example]

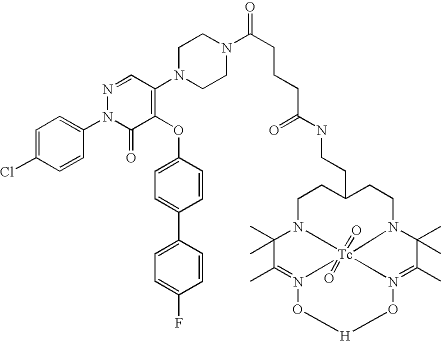

16 μg stannous chloride dehydrate, 25 μg methylene diphosphonic acid, 4500 μg sodium hydrogen carbonate, 600 μg sodium carbonate, and 200 μg sodium p-aminobenzoate are added to an aqueous solution of 50 μg Precursor 3. 1 ml (˜500 MBq) of ^{99m}TcO₄₋ is added and the resultant solution is left to stand at room temperature for 30 minutes, followed by analysis by HPLC and/or TLC using the following conditions:

| | | |
|---|---|---|
| HPLC | Column: | Phenomenex Gemini, 5 u, 4.6 × 150 mm |
| | UV: | 220 nm |
| | Flow: | 1 ml/min |
| | Eluent A: | 0.2% aqueous ammonia (made up using |
| | | 0.88 ammonia) |
| | Eluent B: | acetonitrile |
| | Gradient: | 10-60% B over 10 mins |

| | |
|---|---|
| TLC | 1) silica gel strip run in saline |
| | 2) silica gel strip run in 50:50 0.1M NH₄OAc:MeOH. |

### Example 10

### Synthesis of Imaging Agent 13 [Prophetic Example]

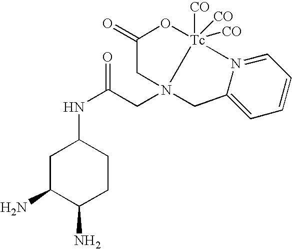

### (i) Synthesis of N-(carboxymethyl)-N-(2-pyridinylmethyl)glycine t-butyl ester (chelator for Tc^{99m}(CO₃)⁺)

This compound is synthesised by a slight modification of the procedure described for the corresponding methyl ester in Stichelberger et al [Nuclear Medicine and Biology (2003) 30(5) 465], by replacing methyl bromoacetate by t-butyl bromoacetate in the synthesis protocol.

### (ii) Conjugation of chelator from Step (i) to 1,2-Diaminocyclohexane

To a solution of 1,2-Diaminocyclohexane (0.50 mmol—synthesis described in Gacheru et al J. Biol. Chem. 1989 264(22) 12963-9), chelator from Step (i) (0.55 mmol) and PyAOP (0.55 mmol) in DMF (5 ml) is added N-methylmorpholine (1.1 mmol). Progress of the reaction is monitored by reverse phase HPLC using a suitable water/acetonitrile (0.1% TFA) gradient. After complete conversion of starting material the solution is concentrated in vacuo and the residue is purified by reverse phase chromatography.

### (iii) Deprotection of Conjugate from Step (ii)

A solution of compound from Step (ii) in TFA/water (95:5) mixture is stirred until complete cleavage of Boc groups and t-butyl ester is confirmed by HPLC analysis. The reaction mixture is concentrated in vacuo and the residue is purified by preparative reverse phase chromatography using a suitable water/acetonitrile (0.1% TFA) gradient.

### (iv) Radiolabelling of Precursor from Step (iii)

Radiolabelling is performed using ^{99m}Tc(H₂O)₃(CO)₃⁺ as described in Psimadas et al [Applied Radiation and Isotopes (2006) 64, 151]. Radiochemical analysis is performed by reverse phase HPLC using a suitable water/methanol (0.1% TFA) gradient.

### Example 11

### Synthesis of Non-Radioactive Imaging Agent 9

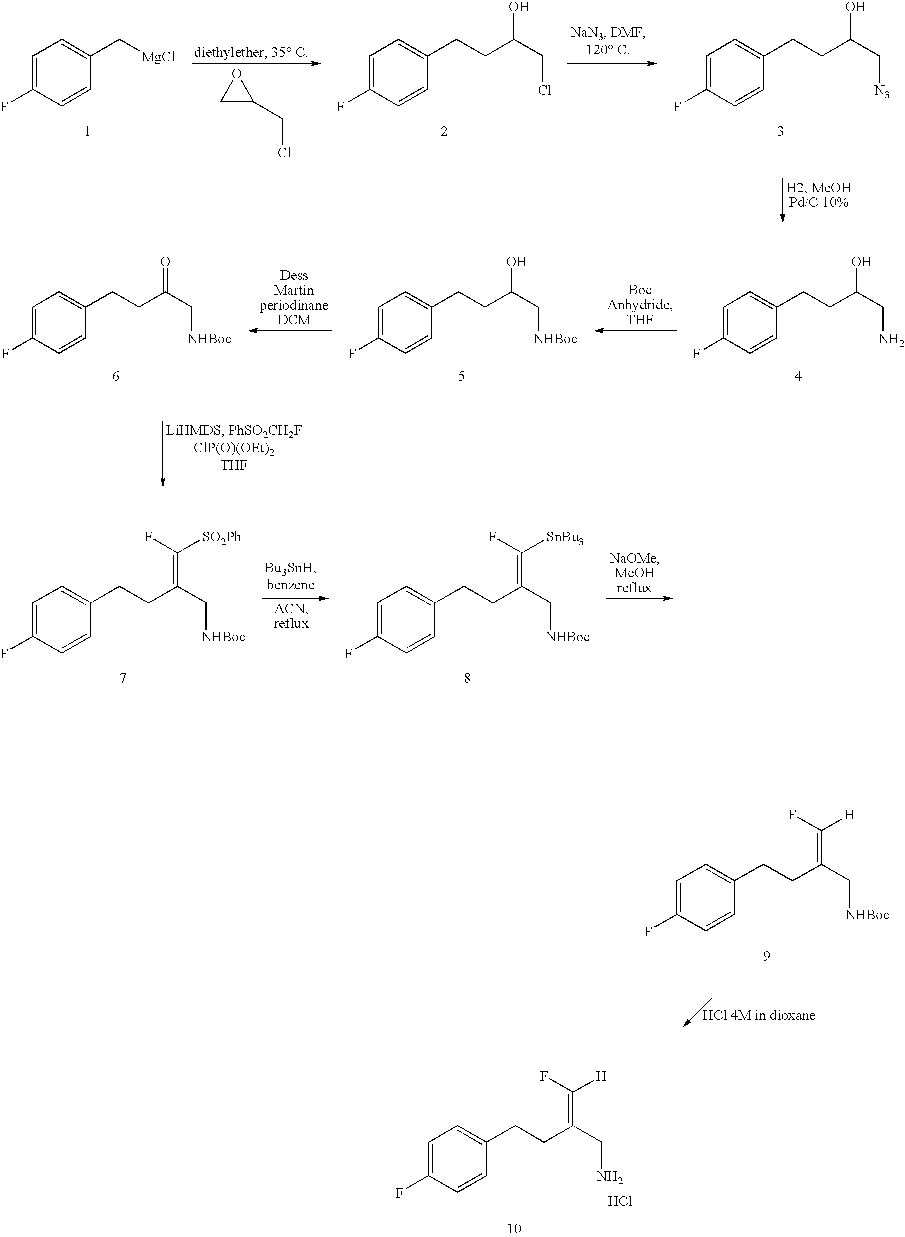

### (i) Preparation of Compound 2

Epichlorohydrin (3.85 g, 0.0417 mmoles, 1 eq) was slowly added to a 0.25M THF solution of the commercially available Grignard salt 1 (0.0375 mmoles, 0.9 eq). The solution was stirred at 35° C. for about two hours before the reaction went to completion. (TLC: ethylacetate/petroleum ether, 2:8). Water was slowly added to the reaction mixture. The precipitated magnesium salt was filtered off and the filtrate concentrated under reduced pressure to remove the THF. The remaining water solution was washed with DCM (×3). The organic layer was dried over anhydrous magnesium sulphate, the magnesium sulphate was filtered off and the solvent was evaporated under reduced pressure. The crude product was then purified by chromatography (10 to 20% ethyl acetate in petroleum ether). Yield: 70%.

### (ii) Preparation of Compound 3

Compound 2 (1 g, 0.005 moles, 1 eq) was dissolved in DMF (25 ml) and sodium azide (1.6 g, 0.025 moles, 5 eq) was added. The reaction mixture was left stirring overnight at 120° C. (TLC: ethylacetate/petroleum ether, 2:8). Water was added and the solution was extracted with diethylether (×2). The organic layer was dried over anhydrous magnesium sulphate, the magnesium sulphate was filtered off and the solvent was evaporated under reduced pressure to a volume of about 20 ml. Then, ethylacetate was added and the solution was again concentrated to a volume of about 5 ml. This residue was then submitted to purification by chromatography (5 to 20% ethyl acetate in petroleum ether). Yield: 90%.

(iii) Preparation of Compound 4

To a solution of 3 (1.035 g, 0.005 mmoles) in methanol 10% palladium on charcoal (200 mg) was added. The mixture was then submitted to hydrogenation overnight using the Parr apparatus. After this time, the catalyst was filtered off and the filtrate was evaporated to dryness under reduced pressure affording a yellowish oil, which was identified as the desired amine 4 by ¹H-NMR. Yield: 95%.

### (iv) Preparation of Compound 5

Boc-anhydride (1.414 g, 6.48 mmoles, 1.1 eq) was added to a solution of 4 (1.078 g, 5.89 mmoles, 1 eq) in THF at 0° C. The solution was then left stirring overnight. The THF was evaporated and the residue was taken into water. The water was extracted with ethylacetate (×3). The organic layer was dried over anhydrous magnesium sulphate, the magnesium sulphate was filtered off and the solvent was evaporated under reduced pressure to dryness to afford a yellow oil. This was then submitted to purification by chromatography (20 to 70% ethyl acetate in petroleum ether). Yield: 50%.

### (v) Preparation of Compound 6

A solution of the alcohol 5 (1.3 g, 4.6 mmoles, 1 eq) in DCM at 0° C. was treated with pyridine (820 μl, 10.12 mmoles, 2.2 eq) followed by addition of Dess-Martin periodinane (3.90 g, 9.2 mmoles, 2 eq). The reaction was then left stirring at room temperature (TLC: petroleum ether/ethylacetate, 3:7). After about 3 hours, few drops of water were added and the reaction was quenched with saturated aqueous sodium bicarbonate and saturated sodium sulphite and extracted with DCM (×3). The organic layer was dried over anhydrous magnesium sulphate, the magnesium sulphate was filtered off and the solvent was evaporated under reduced pressure to dryness to afford a gum. This was submitted to purification by chromatography (20 to 50% ethyl acetate in petroleum ether). Yield: 40%.

### (vi) Preparation of Compound 7

To a solution of fluoromethyl phenyl sulphone (297 mg, 1.708 mmoles, 2 eq) and diethylchlorophosphate (247 μl, 1.708 mmoles, 2 eq) in THF under nitrogen and at −60° C., LiHDMS 1.0M in THF (3.42 ml, 3.416 mmoles, 4 eq) was added. The solution was left stirring for about 30 minutes and the ketone, dissolved in THF, was added. The reaction was left stirring at rt for 12 hours (TLC ethylacetate/petroleum ether, 3:7). Reaction mixture was submitted to purification by chromatography 15 to 40% ethylacetate in petroleum ether). NMR confirmed the major product to be the desired compound 7. Yield: 71.4%.

(vii) Preparation of Compound 8

Compound 7 (267 mg, 0.61 mmoles, 1 eq), tributyltin hydride (533 mg, 1.83 mmoles, 3 eq) and ACN (15 mg, 0.061 mmoles, 0.1 eq) were dissolved in benzene and the mixture was refluxed overnight (TLC ethylacetate/petroleum ether, 1:9). The benzene was evaporated under reduced pressure and the crude was analysed by NMR, which showed the desired product to be the major component. The mixture was then submitted to purification by chromatography using 1 to 30% ethylacetate in petroleum ether. Yield: 54%.

(viii) Preparation of Compound 9

To a solution of compound 8 in THF, NaOMe 0.5M in methanol was slowly added and the reaction mixture was heated to 60° C. for 12 hours (TLC ethylacetate/petroleum ether, 1:9.). The solvent was removed under reduced pressure and the crude was purified by chromatography using ethylacetate 10 to 30% in petroleum ether. Yield: 72%.

### (ix) Preparation of Compound 10 [Non-Radioactive Imaging Agent 9]

Compound 9 (70 mg, 0.236 mmoles, 1 eq) was dissolved in a solution of 4M HCl in dioxane (1 ml, ˜4 eq). The reaction mixture was stirred at room temperature overnight. The solvent was removed under reduced pressure. Diethylether was added to the clear oil and the product precipitated as a white solid. ¹H-NMR and NOE experiments on the pure compound confirmed 10 to be the desired compound and the desired isomer (fluorine is trans to the amine).

### Example 12

### Synthesis of 5-(4-ethyl-piperazin-1-yl)-4-(4′-hydroxy-biphenyl-4-yloxy)-2-p-tolyl-2H-pyridazin-3-one [Precursor 5]

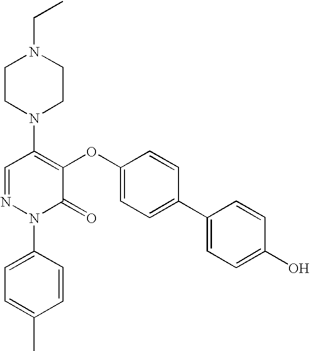

The reaction was carried out in a glass vial for microwave reactions. 4,4′-Dihydroxy biphenyl (Fluka, 11 mg, 0.060 mmole) was added to a suspension of cesium carbonate (Fluka, 39 mg, 0.12 mmole) in dry N,N-dimethylformamide (Rathburn, 2 mL) under argon. After one hour the mixture was added to 4-chloro-5-(4-ethyl-piperazin-1-yl)-2-p-tolyl-2H-pyridazin-3-one (20 mg, 0.060 mmole). The mixture was heated by microwave irradiation at 130° C. for 9 hours. N,N-dimethylformamide was evaporated under reduced pressure and the reaction mixture purified using reversed phase HPLC (column Phenomenex Luna C18(2) 5μ 21.2×250 mm, solvents: A=water/0.1%/0.1 TFA and B=acetonitrile/0.1% TFA; gradient 2-40% B over 60 min; flow 10 ml/min, UV detection at 214 and 254 nm) affording 1.4 mg of pure compound. HPLC analysis (column Phenomenex Luna C18(2) 5μ 4.6×250 mm, solvents: A=water/0.1% TFA and B=acetonitrile/0.1% TFA; gradient 20-40% B over 20 min; flow 1.0 ml/min, UV detection at 214 and 254 nm, t_{R}=19.8 min) and LC-MS analysis (column Phenomenex Luna C18(2) 3 μm 2.0×20 mm, solvents: A=water/0.1% TFA and B=acetonitrile/0.1% TFA; gradient 10-80% B over 5 min; flow 0.6 ml/min, UV detection at 214 and 254 nm, ESI-MS; t_{R}=2.5 min, m/z 483.3 (MH+)) confirmed the product.

What is claimed is:

1) An imaging agent comprising:
(i) a lysyl oxidase (LOX) binder; and,
(ii) an imaging moiety
wherein said imaging moiety is either an integral part of the LOX binder or is conjugated to the LOX binder via a suitable chemical group.

2) The imaging agent of claim 1 wherein said LOX binder is selected from:
(i) a homocysteine lactone;
(ii) a pyridazinone;
(iii) a halogenated allylamine; and,
(iv) a vicinal diamine.

3) The imaging agent of claim 2 wherein said LOX binder is a homocysteine lactone and is of Formula I: 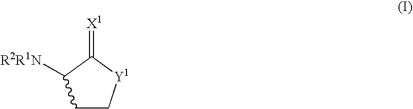
wherein
R¹ and R² are independently selected from the group consisting of hydrogen, an amino acid residue, C₁₋₆ alkyl, halo, C₁₋₆ haloalkyl, hydroxyl, C₁₋₆ hydroxyalkyl, C₁₋₆ alkoxyl, C₂₋₆ alkoxyalkyl, C₁₋₆ acyl, C₂₋₆ alkacyl, C₁₋₆ carboxyl, C₂₋₆ carboxyalkyl, amino, C₁₋₆ alkylamino, nitro, cyano, and thiol; and,
X¹ and Y¹ are independently selected from S, Se or O.

4) The imaging agent of claim 3 wherein:
R¹ and R² are independently selected from the group consisting of hydrogen, an amino acid residue, C₁₋₆ alkyl, C₁₋₆ haloalkyl, C₁₋₆ hydroxyalkyl, C₂₋₆ alkoxyalkyl, C₂₋₆ carboxyalkyl or C₁₋₆ alkylamino.

5) The imaging agent of claim 3 wherein:
R¹ is hydrogen and R² is an amino acid residue or C₁₋₆ alkylamino.

6) The imaging agent of claim 3 wherein said homocysteine lactone is selected from:
(i) glycylhomocysteine thiolactone,
(ii) β-alanylhomocysteine thiolactone,
(iii) γ-aminobutyrylhomocysteine thiolactone,
(iv) ε-aminocaproylhomocysteine thiolactone,
(v) lysylhomocysteine thiolactone.

7) The imaging agent of claim 2 wherein said LOX binder is a pyridazinone and is of Formula II: 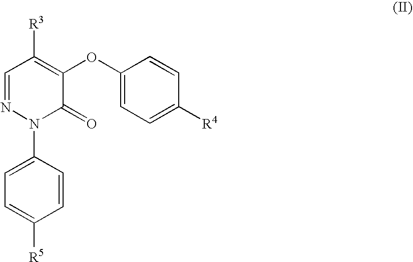
wherein:
one of R³ and R⁴ is X² and the other is Y² wherein;
X² is a 5- or 6-membered nitrogen-containing aliphatic or aromatic ring substituted with 0-4 substituents selected from C₁₋₆ alkyl, C₁₋₆ hydroxyalkyl, C₁₋₆ sulphonyl and imidazolyl; and,
Y² is a phenyl group with 0-4 substituents selected from C₁₋₆ alkyl, hydroxyl, halo, C₁₋₆ aminoalkyl, and C₁₋₆ alkylamido;
R⁵ is methyl or chloro.

8) The imaging agent of claim 7 wherein:
X² is pyrroyl, imidazoyl, pyrazoyl, piperidyl or piperazyl with 0-2 substituents selected from C₁₋₆ alkyl, C₁₋₆ hydroxyalkyl and C₁₋₆ sulphonyl.

9) The imaging agent of 7 wherein:
X² is imidazoyl, piperidyl or piperazyl substituted with 0-2 substituents selected from C₁₋₆ alkyl, C₁₋₆ hydroxyalkyl and C₁₋₆ sulphonyl; and,
Y² a phenyl group substituted with 0-2 substituents selected from hydroxyl, fluoro, C₁₋₆ aminoalkyl and carbamoyl.

10) The imaging agent of claim 7 wherein said pyridazinone is selected from the compounds of formulae:

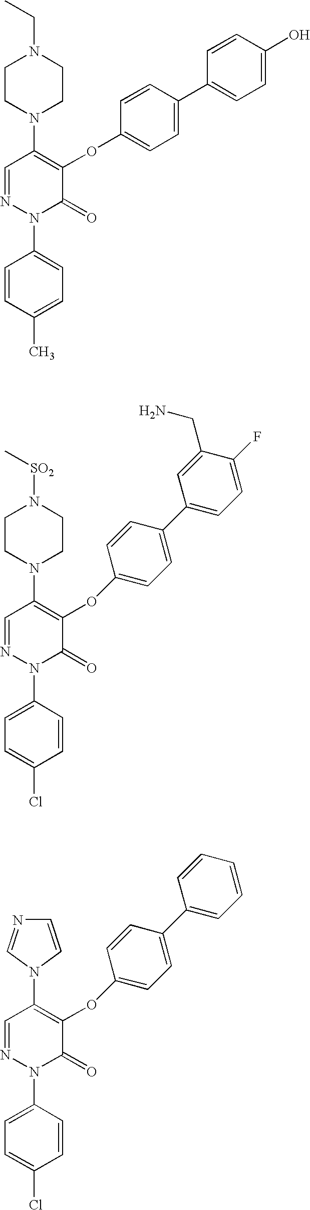

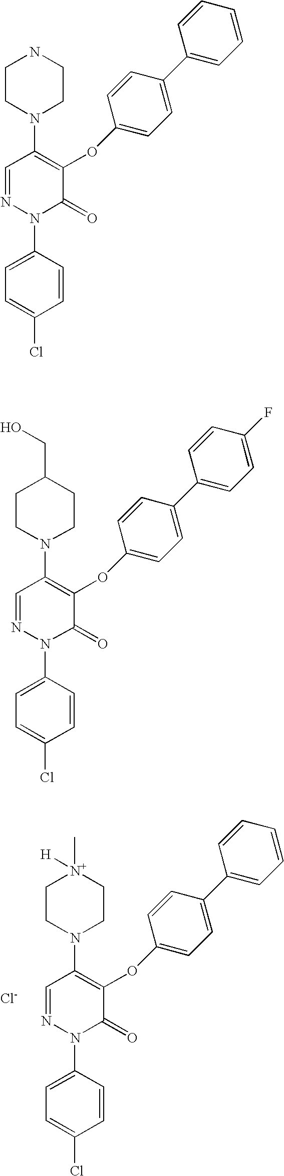

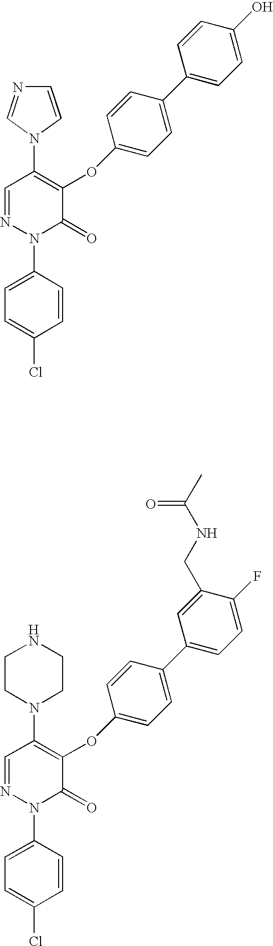

11) The imaging agent of claim 2 wherein said LOX binder is a halogenated allylamine and is of Formula III: 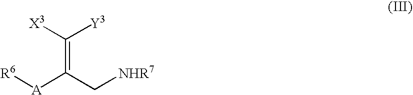
wherein:
R⁶ is methyl, naphthyl, indenyl, fluorenyl, piperidinyl, pyrrolyl, thienyl, furanyl, indolyl, thianaphthylenyl, benzofuranyl, or a phenyl group substituted with 0-4 substituents selected from C₁₋₆ alkyl, C₁₋₆ alkoxy, hydroxyl, chloro, fluoro, bromo, iodo, trifluoromethyl, nitro, C₂₋₆ alkylcarbonyl, benzoyl or phenyl;
R⁷ is hydrogen or C₁₋₆ alkyl;
A is a linker of Formula -(L³)ₚ-
wherein each L³ is independently —CO—, —CR′₂—, —CR′═CR′—, —C≡C—, —CR′₂CO₂—, —CO₂CR′₂—, —NR′—, —NR′CO—, —CONR′—, —NR′(C═O)NR′—, —NR′(C═S)NR′—, —SO₂NR′—, —NR′SO₂—, —CR′₂OCR′₂—, —CR′₂SCR′₂—, —CR′₂NR′CR′₂—, a C₄₋₈ cycloheteroalkylene group, a C₄₋₈ cycloalkylene group, a C₅₋₁₂ arylene group, a C₃₋₁₂ heteroarylene group, an amino acid, a polyalkyleneglycol, polylactic acid or polyglycolic acid moiety;
p is an integer of value 0 to 10;
each R′ group is independently H or C₁₋₁₀ alkyl, C₃₋₁₀ alkylaryl, C₂₋₁₀ alkoxyalkyl, C₁₋₁₀ hydroxyalkyl, C₁₋₁₀ fluoroalkyl, or 2 or more R′ groups, together with the atoms to which they are attached form a carbocyclic, heterocyclic, saturated or unsaturated ring; and,
X³ and Y³ are independently selected from the group consisting of hydrogen, fluoro, chloro and bromo.

12) The imaging agent of claim 11 wherein:
R⁶ is a phenyl group substituted with 0-2 substituents selected from C₁₋₆ alkyl, C₁₋₆ alkoxy, hydroxyl, chloro, fluoro, bromo, iodo, trifluoromethyl, nitro, C₂₋₆ alkylcarbonyl, benzoyl or phenyl;
R⁷ is hydrogen;
A is —(CH₂)_{q}— wherein q is an integer of value 1-6; and,
X³ is hydrogen.

13) The imaging agent of claim 11 wherein:
R⁶ is a phenyl group optionally substituted with 1-2 substituents selected chloro, fluoro, bromo and iodo;
R⁷ is hydrogen;
A is —(CH₂)_{q}— wherein q is an integer of value 1-6; and,
X³ is hydrogen and Y³ is fluoro.

14) The imaging agent of claim 11 wherein said halogenated allylamine is of formula:

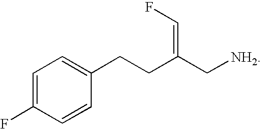

15) The imaging agent of claim 2 wherein the vicinal diamine is of Formula IV: 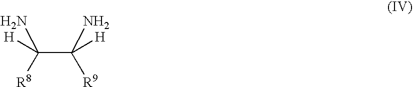
wherein:
R⁸ and R⁹ are each independently hydrogen, C₁₋₆ alkyl, or R⁸ and R⁹ together with the carbons to which they are attached form a 6-14-membered optionally-substituted aliphatic or aromatic ring system.

16) The imaging agent of claim 15 wherein the two primary amines of Formula IV are aligned in the same stereochemical plane.

17) The imaging agent of claim 15 wherein R⁸ and R⁹ together with the carbons to which they are attached form a cyclohexyl or a dicyclohexyl ring optionally-substituted with 1-3 substituents selected from C₁₋₃ alkyl and halo.

18) The imaging agent of claim 1 wherein said imaging moiety is selected from:
(i) a radioactive metal ion;
(ii) a paramagnetic metal ion;
(iii) a gamma-emitting radioactive halogen;
(iv) a positron-emitting radioactive non-metal;
(v) a hyperpolarised NMR-active nucleus;
(vi) a reporter suitable for in vivo optical imaging; and
(vii) a β-emitter suitable for intravascular detection.

19) The imaging agent of claim 18 wherein the imaging moiety is a radioactive metal ion.

20) The imaging agent of claim 19 wherein the radioactive metal ion is ^{99m}Tc.

21) The imaging agent of claim 18 wherein the imaging moiety is a gamma-emitting radioactive halogen.

22) The imaging agent of claim 21 wherein the gamma-emitting radioactive halogen is selected from ¹²³I, and ¹³¹I.

23) The imaging agent of claim 18 wherein the imaging moiety is a positron-emitting radioactive non-metal.

24) The imaging agent of claim 23 wherein the positron-emitting radioactive non-metal is selected from ¹⁸F and ¹¹C.

25) A method for the preparation of the imaging agent of claim 1, which comprises reaction of a precursor with a suitable source of the imaging moiety of said claim, wherein said precursor comprises:
(i) a LOX binder as defined in said claim; and,
(ii) a chemical group capable of reacting with a source of the imaging moiety to give the imaging agent of said claim;
wherein said chemical group is either an integral part of said LOX binder, or is conjugated to said LOX binder.

26) The method according to claim 25 wherein said chemical group:
(i) comprises a chelator capable of complexing a metallic imaging moiety;
(ii) comprises an organometallic derivative such as a trialkylstannane or a trialkylsilane;
(iii) comprises a derivative containing an alkyl halide, alkyl tosylate or alkyl mesylate for nucleophilic substitution;
(iv) comprises a derivative containing an aromatic ring activated towards nucleophilic or electrophilic substitution;
(v) comprises a derivative containing a functional group which undergoes facile alkylation; or,
(vi) comprises a derivative which alkylates thiol-containing compounds to give a thioether-containing product

27) The method according to claim 25
wherein said precursor is in sterile, a pyrogenic form.

28) The method according to claim 25
wherein the precursor is bound to a solid phase.

29) A precursor as defined in the method of claim 25 wherein said chemical group:
(i) comprises a chelator capable of complexing a metallic imaging moiety;
(ii) comprises an organometallic derivative such as a trialkylstannane or a trialkylsilane;
(iii) comprises a derivative containing an alkyl halide, alkyl tosylate or alkyl mesylate for nucleophilic substitution;
(iv) comprises a derivative which alkylates thiol-containing compounds to give a thioether-containing product

30) A pharmaceutical composition comprising the imaging agent of claim 1 together with a biocompatible carrier, in a form suitable for human administration.

31) The pharmaceutical composition of claim 30 wherein said imaging agent comprises a radioactive imaging moiety.

32) The pharmaceutical composition of claim 31, which has a radioactive dose suitable for a single patient and is provided in a suitable syringe or container.

33) A kit for the preparation of the pharmaceutical composition of claim 30 which comprises a precursor comprising:
(a) a lysyl oxidase (LOX) binder; and,
(b) a chemical group capable of reacting with a source of the imaging moiety to give the imaging agent in said claim wherein the imaging moiety is either an integral part of the LOX binder or is conjugated to the LOX binder via a suitable chemical group.

34) An imaging agent of claim 1 for use in an in vivo diagnostic or imaging method.

35) The imaging agent of claim 34 wherein said method relates to the in vivo imaging of a condition in which LOX is upregulated.

36) The imaging agent of claim 35 wherein the condition in which LOX is upregulated is a condition associated with fibrosis.

37) The imaging agent of claim 36 wherein said condition associated with fibrosis is liver fibrosis, congestive heart failure, glomerulosclerosis or respiratory failure.

38) The imaging agent of claim 37 wherein said condition associated with fibrosis is liver fibrosis.

39) A method for the in vivo diagnosis or imaging in a subject of a condition in which LOX is upregulated, comprising administration of the pharmaceutical composition of claim 30.

40) Use of the imaging agent of claim 1 for imaging in vivo in a subject of a condition in which LOX is upregulated wherein said subject is previously administered with the pharmaceutical composition of said claim together with a biocompatible carrier, in a form suitable for human administration.

41) Use of the imaging agent of claim 1 for the manufacture of a pharmaceutical for the imaging in vivo of a condition in which LOX is upregulated.

42) A method of monitoring the effect of treatment of a human or animal body with a drug to combat a condition in which LOX is upregulated, said method comprising administering to said body the imaging agent of claim 1 and detecting the uptake of said imaging agent.
